(19) United States
(12) Patent Application Publication
(10) Pub. No.: US 2022/0172643 A1
(43) Pub. Date: Jun. 2, 2022

(54) INFORMATION PROCESSING DEVICE

(71) Applicant: Masaaki TAKAYAMA, Osaka (JP)

(72) Inventor: Masaaki TAKAYAMA, Osaka (JP)

(21) Appl. No.: 17/602,531

(22) PCT Filed: Apr. 10, 2020
(86) PCT No.: PCT/JP2020/016147

§ 371 Date: Oct. 8, 2021

(30) Foreign Application Priority Data
Apr. 10, 2019 (JP) 2019-075145

(51) Int. Cl. G09B 19/00; G16H 50/30; G16H 20/00; G16H 70/20

(52) U.S. Cl. CPC G09B 19/0084; G16H 70/20; G16H 20/00; G16H 50/30

(57) ABSTRACT

The present invention addresses the problem of enabling a physician to efficiently guide a patient to an examination and enabling the patient to efficiently perform self-care. An oral health age management section 107 manages dental health of a patient U according to an oral health age of the patient U indicated by a physician D, assuming a dental examination of the patient U by the physician D. An oral health score management section 108 manages the dental health of the patient U according to an oral health score given to the patient U according to the self-care practiced by the patient U. A guidance section 109 performs guidance for the patient U to receive an examination according to the oral health score given to the patient U. Therefore, the above problem is solved.

### TECHNICAL FIELD

The present invention relates to an information processing device.

### BACKGROUND ART

There are technologies that support a user in receiving a test or a physical examination (Patent Literature 1) and give advice for self-care (Patent Literature 2).

### CITATION LIST

### Patent Literature

### [Patent Literature 1]

Japanese Patent Application Laid-Open Publication No. 2018-81397

### [Patent Literature 2]

Japanese Patent Application Laid-Open Publication No. 2019-200555

### SUMMARY OF INVENTION

### Technical Problem

However, in this field, there is the need for development of technology which enables physicians to efficiently guide patients to examinations and enables patients to efficiently perform self-care.

The present invention takes these circumstances into account, and an object thereof is to enable a physician to efficiently guide a patient to an examination and to enable the patient to efficiently perform self-care.

### Solution to Problem

To achieve the above object, an information processing device according to an aspect of the present invention includes:

a first management means which manages dental health of a patient according to a first evaluation result based on a first index assuming predetermined treatment to improve the dental health of the patient;

a second management means which manages the dental health of the patient according to a second evaluation result based on a second index; and

a guidance means which performs predetermined guidance for the patient to perform the predetermined treatment to improve the dental health of the patient according to the second evaluation result.

### Advantageous Effects of Invention

According to the present invention, a physician can efficiently guide a patient to an examination and enable the patient to efficiently perform self-care.

### BRIEF DESCRIPTION OF DRAWINGS

FIG. 1 is a diagram illustrating an overview of an example of services applicable during a patient visit, among services which can be implemented by an information processing system including a server according to a first embodiment of the information processing device of the present invention.

FIG. 2 is a diagram illustrating an overview of an example of a service which monitors procedure of a patient, among the present services.

FIG. 3 is a diagram illustrating the overview of the example of the service which monitors the procedure of the patient, among the present services.

FIG. 4 is a diagram illustrating an overview of an example of a service applicable during an examination, among the present services.

FIG. 5 is a diagram illustrating an overview of an example of a service applicable during settlement processing, among the present services.

FIG. 6 is a diagram illustrating an overview of an example of a service applicable during evaluation of a physician or the like by a patient, among the present services.

FIG. 7 is a diagram illustrating an overview of an example of various services which may be provided based on the evaluation result of the physician or the like by the patient in FIG. 6, among the present services.

FIG. 8A is a diagram illustrating an example of a top page as a specific example of an image displayed by a patient terminal of the patient using the services in FIG. 7.

FIG. 8B is a diagram illustrating an example of a medical institution search screen as a specific example of an image displayed by the patient terminal of the patient using the services in FIG. 7.

FIG. 9A is a diagram illustrating an example of a medical institution introduction as a specific example of an image displayed by the patient terminal of the patient using the services in FIG. 7.

FIG. 9B is a diagram illustrating an example of medical institution information as a specific example of an image displayed by the patient terminal of the patient using the services in FIG. 7.

FIG. 10A is a diagram illustrating an example of medical institution evaluation information as a specific example of an image displayed by the patient terminal of the patient using the services in FIG. 7.

FIG. 10B is a diagram illustrating an example of a screen displayed when an appointment is made by telephone as a specific example of an image displayed by the patient terminal of the patient using the services in FIG. 7.

FIG. 11A is a diagram illustrating an example of medical institution and physician evaluation information as a specific example of an image displayed by the patient terminal of the patient using the services in FIG. 7.

FIG. 11B is a diagram illustrating an example of physician evaluation information as a specific example of an image displayed by the patient terminal of the patient using the services in FIG. 7.

FIG. 12 illustrates a configuration of the information processing system applied to the present services in FIG. 7, that is, the information processing system including the server according to the first embodiment of the information processing device of the present invention.

FIG. 13 is a block diagram illustrating an example of a hardware configuration of the server in the information processing system illustrated in FIG. 12.

FIG. 14 is a functional block diagram illustrating an example of a functional configuration for implementing evaluation support processing within the functional configuration of the server in FIG. 13 in the information processing system in FIG. 12.

FIG. 15A is a diagram illustrating an example of a patient registration card displayed by the patient terminal as a specific example of the services illustrated in FIG. 7.

FIG. 15B is a diagram illustrating an example of a medical care history displayed by the patient terminal as a specific example of the services in FIG. 7.

FIG. 16A is a diagram illustrating an example of an evaluation entry screen displayed by the patient terminal as a specific example of the services in FIG. 7.

FIG. 16B is a diagram illustrating an example of a coupon displayed by the patient terminal as a specific example of the services in FIG. 7.

FIG. 17A is a diagram illustrating an example of retained points displayed by the patient terminal as a specific example of the services in FIG. 7.

FIG. 17B is a diagram illustrating an example of point exchange displayed by the patient terminal as a specific example of the services in FIG. 7.

FIG. 18 is a diagram illustrating an example of an appointment management screen displayed by a physician terminal as a specific example of the services in FIG. 7.

FIG. 19 is an image diagram illustrating an overview of usage suspension measures by a service provider of the services in FIG. 7.

FIG. 20 is a diagram illustrating a specific example of a reception method when a patient visits the medical institution in the services in FIG. 7.

FIG. 21 is a diagram illustrating an overview of an example of a service for guiding a patient to an examination by a physician of a medical facility at which the services of FIGS. 1 to 7 are applied.

FIG. 22 is a functional block diagram illustrating an example of a functional configuration for implementing guidance support processing within the functional configuration of a server in an information processing system according to a second embodiment. This functional configuration enables application of the service in FIG. 21.

FIG. 23A is a diagram illustrating an example of an oral health age inquiry screen displayed by a patient terminal as a specific example of the service in FIG. 21.

FIG. 23B is a diagram illustrating an example of an oral health score inquiry screen displayed by the patient terminal as a specific example of the services in FIG. 7.

FIG. 24A is a diagram illustrating a specific example of an age measurement app.

FIG. 24B is a diagram illustrating a specific example of the age measurement app.

FIG. 24C is a diagram illustrating a specific example of the age measurement app.

FIG. 24D is a diagram illustrating a specific example of the age measurement app.

### DESCRIPTION OF EMBODIMENTS

The following describes embodiments of the present invention using the accompanying drawings.

### First Embodiment

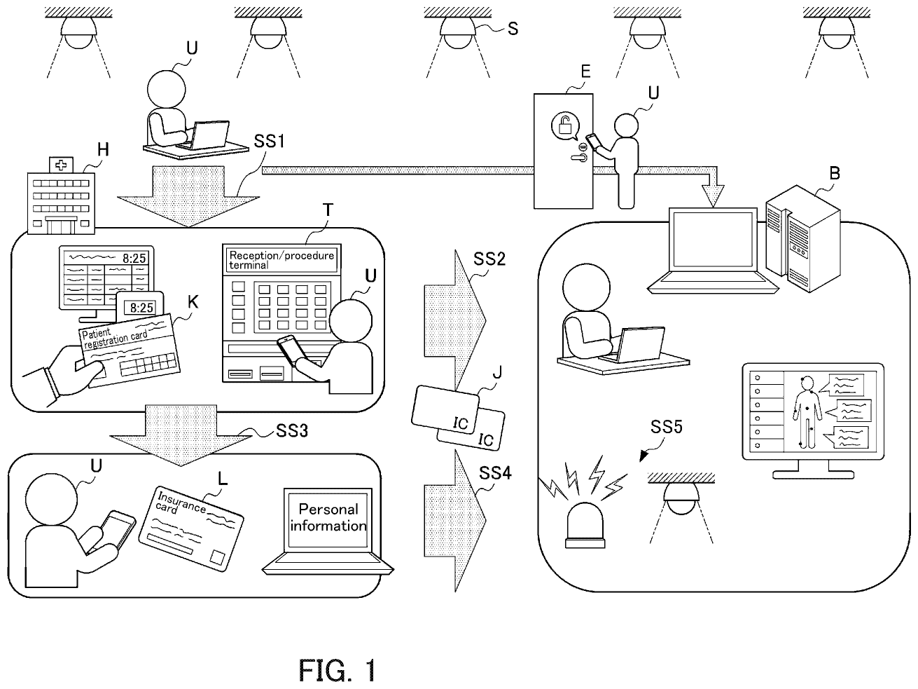

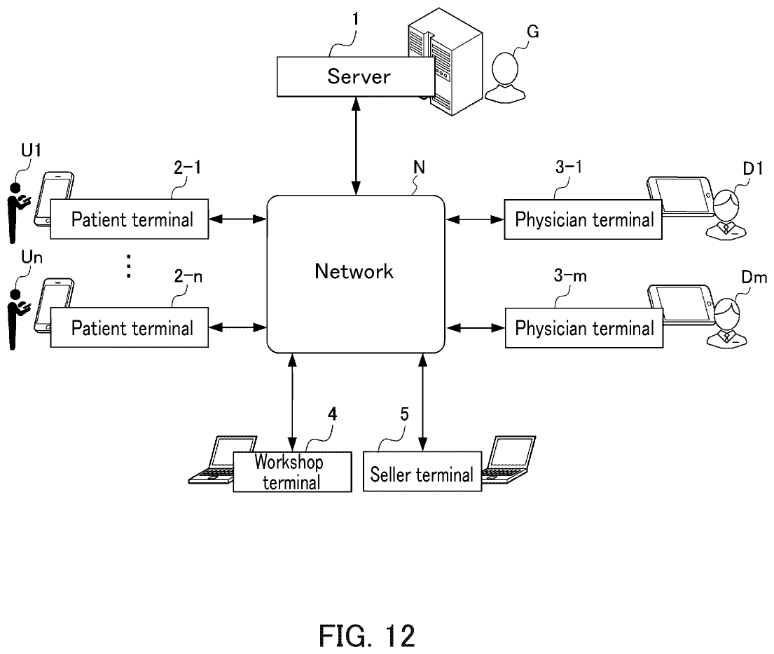

First, an overview of services of an information processing system (refer to FIG. 12 as later described) to which the present invention is applied is described with reference to FIGS. 1 to 7.

FIG. 1 is a diagram illustrating an overview of an example of services applicable during a patient visit, among services (referred to in the following as the “present services”) which can be implemented by an information processing system including a server according to a first embodiment of the information processing device of the present invention.

When a patient U visits (Step SS1), a sensor/card detector S installed at an entrance E of a medical facility H detects the visit and a reception/procedure terminal T starts reception processing.

When the patient U is a returning patient, restriction processing is performed on an ID or the like of the patient U detected from a smartphone or the like of the patient U. When the returning patient U inserts a previously issued patient registration card K into the reception/procedure terminal T, information saved on the patient registration card K is read and a queue card J is issued (Step SS2).

When the patient U is a new patient, the reception/procedure terminal T reads data (personal information) saved on an insurance card L (Step SS3). The read data (personal information) is sent to a medical server B from the reception/procedure terminal T and the patient U is registered (Step SS4).

When the patient U is registered, the reception/procedure terminal T issues a patient registration card K and a queue card J each equipped with an IC chip. Data such as the name of the patient U and an ID capable of uniquely specifying the patient U is saved to the patient registration card K issued by the reception/procedure terminal T. When the patient U does not receive the patient registration card K and the queue card J after a predetermined time has elapsed from the issuance of the patient registration card K and the queue card J, the reception/procedure terminal T emits a warning sound and displays a message to that effect on a screen until the patient U receives the patient registration card K and the queue card J (Step SS5).

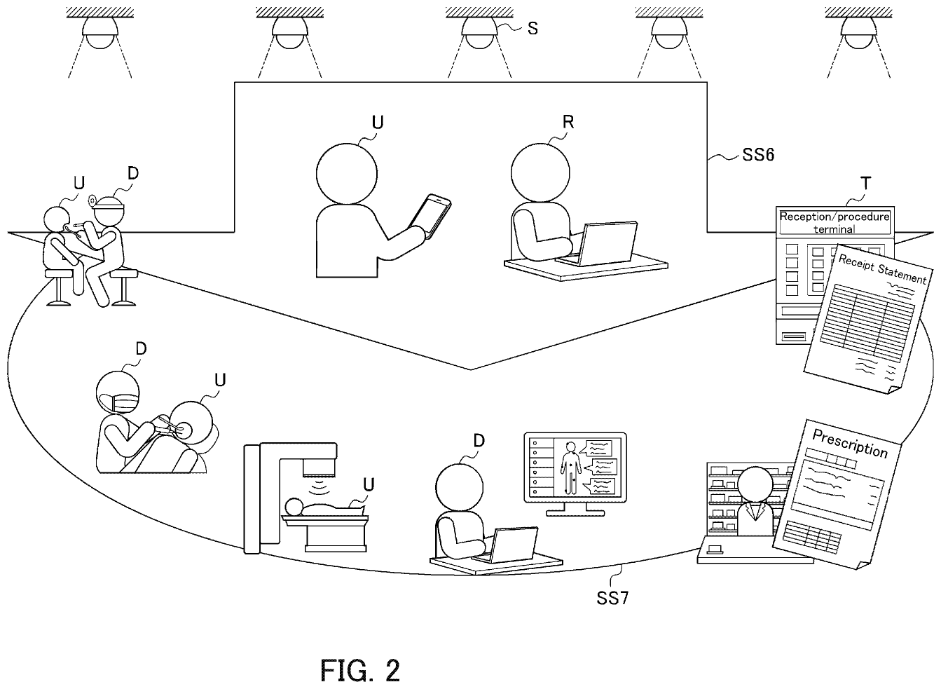

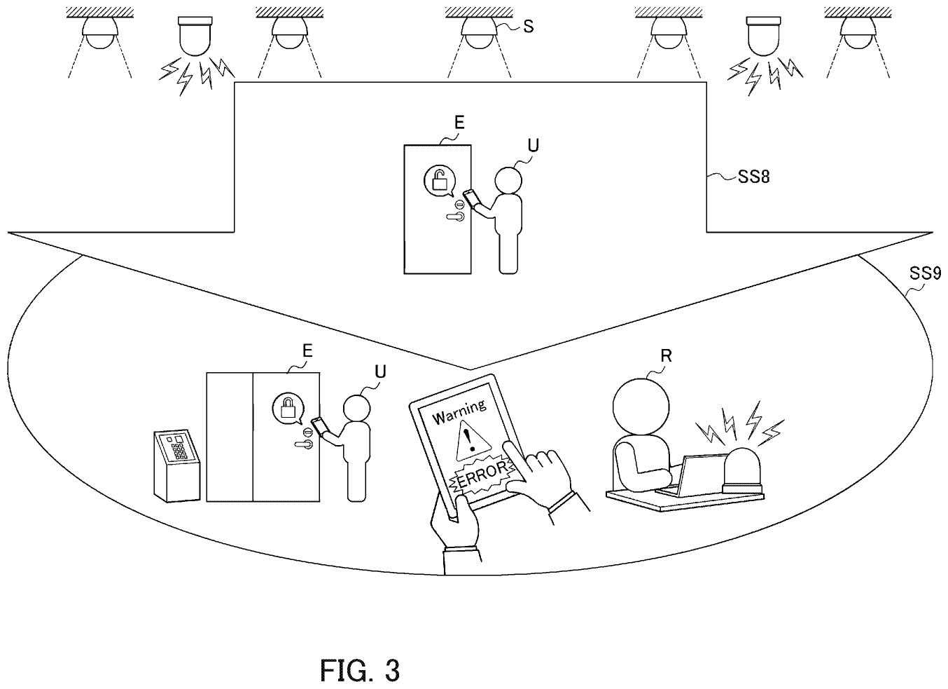

FIGS. 2 and 3 are diagrams illustrating an overview of an example of a service which monitors a procedure of a patient, among the present services.

In the present services as illustrated in FIG. 2, movement of the position of the queue card J detected by the sensor/card detector S is monitored when the queue card J is issued by the reception/procedure terminal T (Step SS6). Furthermore, flags indicating the completion of various hospital procedures (including examination, treatment, testing, input to a medical care terminal, prescription, and payment) are monitored (Step SS7). When the various procedures are not performed in a predetermined order, a predetermined warning is issued. Here, the various hospital procedures may be led or guided by application software (referred to in the following as a “patient app”) dedicated to the present services that is installed to a smartphone or the like of the patient U.

As illustrated in FIG. 3, when all the various scheduled hospital procedures have been completed as a result of detection by the sensor and card detector, a locked entrance is unlocked upon the patient U trying to leave the medical facility (Step SS8). By contrast, when all the various scheduled hospital procedures have not been completed as a result of the detection by the sensor and card detector, the entrance remains locked upon the patient U trying to leave the medical facility and the patient U cannot leave. Furthermore, a predetermined warning (warning to the smartphone or the like of the patient U and notification to the hospital staff) is also issued (Step SS9).

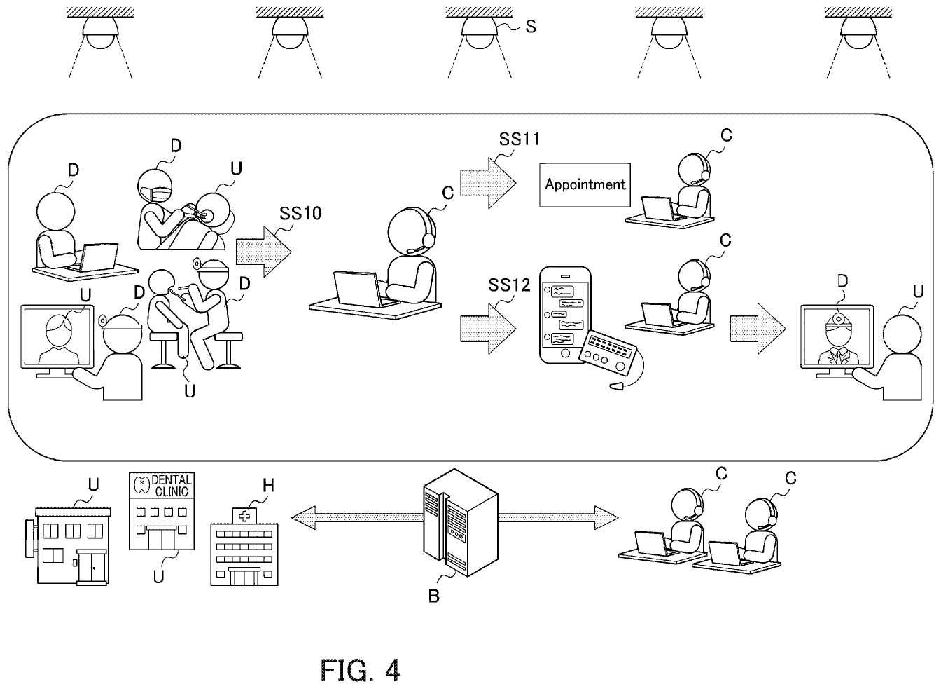

FIG. 4 is a diagram illustrating an overview of an example of a service applicable during an examination, among the present services.

In the present services as illustrated in FIG. 4, when a hospital receptionist R or a physician D is unable to answer the telephone in response to an appointment change or an inquiry by telephone to the medical facility H, the call is transferred to a call center C for reception (Step SS10).

In the call center C, appointment changes or cancellations are received (Step SS11). If necessary, a message is sent to the physician D, or the call is switched to an available physician D (Step SS12).

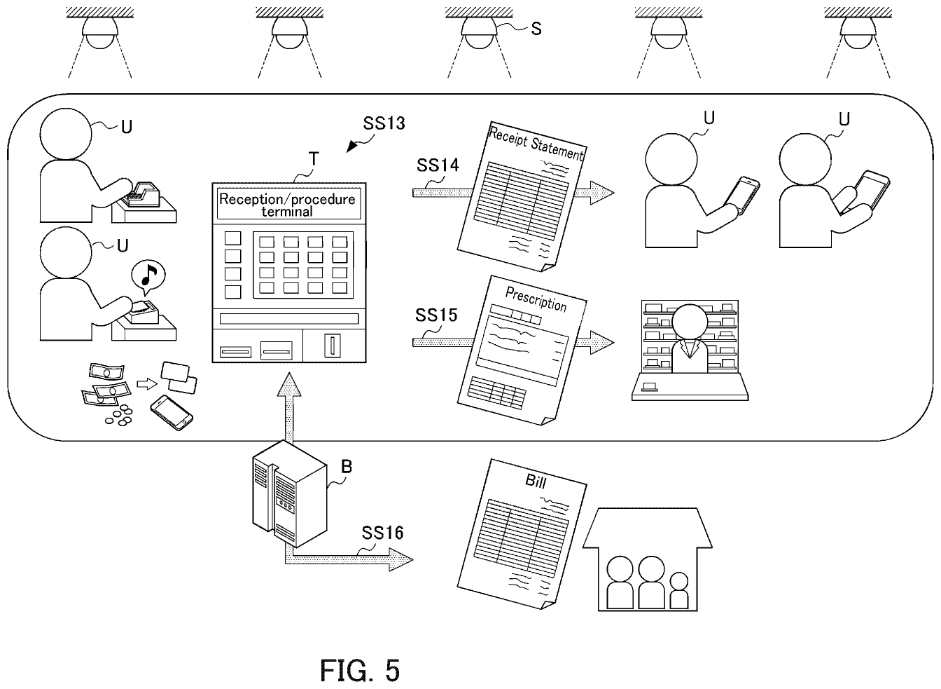

FIG. 5 is a diagram illustrating an overview of an example of a service applicable during settlement processing, among the present services.

In the present services as illustrated in FIG. 5, cashless settlement processing is being implemented. Specifically, processing including registration of next visit and receipt of examination fee is performed as the settlement processing on the reception/procedure terminal (Step SS13). In the processing of the receipt of a medical care fee, it is possible to issue a receipt and make an appointment for the next examination upon confirmation of payment of the examination fee (Step SS14). When medication is being prescribed, it is possible to issue a prescription slip and make an appointment for the next examination (Step SS15).

Here, when the patient U is a child or has a guardian, the settlement processing may be performed by a person responsible for payment and not the patient U themselves (Step SS16). In this case, the settlement processing may be performed not only in the medical facility H but also elsewhere, such as at a remote location.

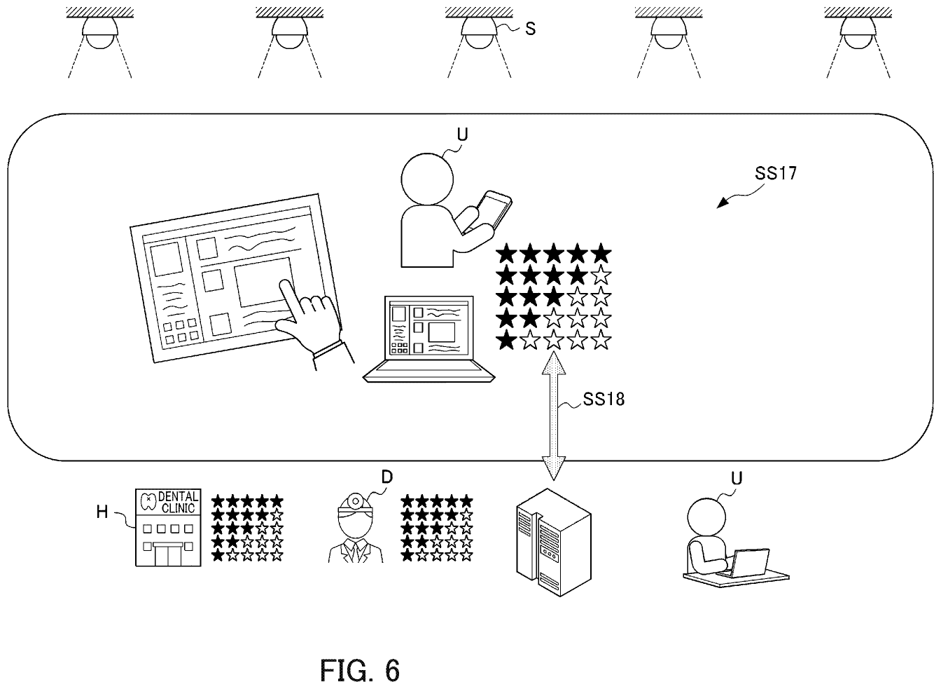

FIG. 6 is a diagram illustrating an overview of an example of a service applicable during evaluation of a physician or the like by a patient, among the present services.

In the present services as illustrated in FIG. 6, evaluation of an examination by the patient U is received after the examination. Specifically, the patient U evaluates the examination using the patient app (Step SS17).

Subjects to be evaluated include, for example, an overall evaluation, an evaluation of the physician in charge, and an evaluation of a receptionist or an assistant. Evaluated items of the overall evaluation include evaluation of the entire medical facility. Evaluated items of the evaluation of the physician D in charge include attitude toward the patient U, cleanliness, examination content, and examination time. Evaluated items of the evaluation of the receptionist or the assistant include attitude toward the patient U, cleanliness, and the content of the work they are responsible for.

For example, a five-point evaluation (very good, good, average, poor, very poor) is adopted as an evaluation method. The content of the evaluation by the patient U is recorded (Step SS18) and utilized as information subject to an information search by a patient U or for a matching service between a patient U and a physician D.

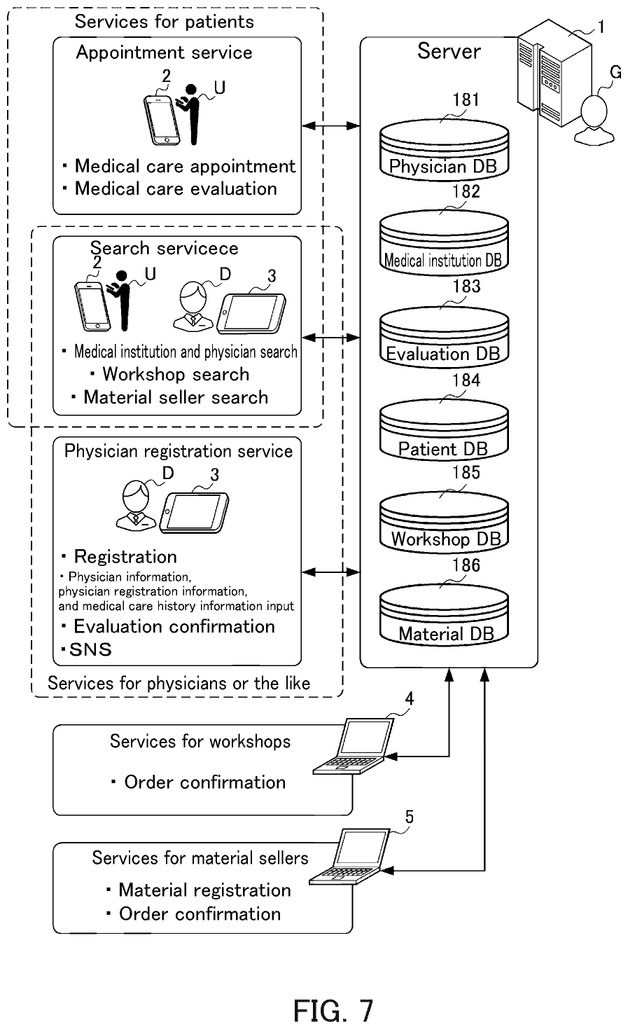

FIG. 7 is a diagram illustrating an overview of an example of various services provided based on the evaluation result of a physician or the like by the patient in FIG. 6, among the present services.

The various services provided based on the result of items such as the evaluation of the physician D by the patient U in FIG. 6 include services such as services for patients, services for physicians or the like, services for workshops, and services for material sellers as illustrated in FIG. 7.

Services for patients are services to support patient U. Specific examples include an appointment service and a search service.

The appointment service among the services for patients is a service which supports the patient U in making an appointment to receive medical care.

According to the appointment service, the patient U uses the patient app to register for the present services. The patient U can then easily make an appointment by inputting information such as the medical institution or the physician D from whom the patient U wishes to receive medical care, an appointment time, and medical care content using the patient app.

In registering for the present services, the patient U inputs items such as a captured image, a name, a gender, a date of birth, a postal code, an address, a telephone number, and an occupation of the patient U, information uniquely identifying the patient U, and a password as information about the patient U (referred to in the following as “patient information”). The patient information of the patient U is stored and managed in a patient DB 184 of a server 1. Here, an email address or the like may be set as the ID and a medical record number may be linked with the ID as an example of the information uniquely identifying the patient U.

Furthermore, a “location” or “equipment” pertaining to medical care may be included in the information input when the patient U makes an appointment. Through the above, when the patient U making a reservation is an elderly person living in a facility such as a nursing facility, for example, the patient U can easily request visiting medical care to the facility by a desired physician D. Furthermore, when the patient U needs medical care using special equipment (e.g., medical equipment), the patient U can easily request a physician D or a medical institution fulfilling this condition.

The appointment service also accepts an evaluation of the medical care provided by a physician D by a patient U who has received medical care at the medical institution where the appointment took place. Information about the evaluation (referred to in the following as “evaluation information”) is stored and managed in an evaluation DB 183 of the server 1.

Here, evaluation of items such as evaluation of each item of medical care, general evaluation of the physician D, or other comments about the evaluation are included in the “evaluation information” as an evaluation of the physician D by each patient U. For a specific example, items such as reviews or multiple-point evaluations (e.g., five-point evaluations) of the physician D are included in the evaluation information.

That is, a patient U can input an evaluation of the medical care when making an appointment for reexamination at a medical institution after receiving medical care at the medical institution. Here, other than the evaluation of the medical institution by the patient U, the “evaluation of medical care” may include an evaluation by the patient U of an individual physician D (physician D who provided medical care) belonging to the medical institution.

The evaluation by the patient U may be a requirement for the patient U to make a medical care appointment using the appointment service, or some kind of incentive may be given to the patient U to perform the evaluation. Through the above, the number of evaluations by the patient U can be increased, which can be effectively used as reference information by other patients U.

The search service among the services for patients is a service which supports a patient U in searching for a medical institution or a physician D. The search service allows the patient U to easily search for a medical institution or a physician D that matches or has a high degree of matching the wishes of the patient U by only inputting items such as an area in which the patient U desires medical care or medical equipment that the patient U wishes to use using the patient app.

For a specific example, by designating a request based on one or more factors to search for a physician D or a medical institution, the patient U can obtain information about a physician D or a medical institution that fulfills the request. Examples of a “factor” may include skill (ability), field of specialization, educational background, speaking style, personality, gender, age, and appearance of the physician D.

What the patient U seeks from a physician D differs depending on the values of the patient U. Therefore, the patient U can search for a physician D or a medical institution by selecting one or more factors that match the values of the patient U from a plurality of factors.

For example, factors that can be selected by the patient U include the gender of the physician D and the university from which the physician D graduated. That is, some patients U seek only the skill (ability) of a physician D and do not mind if the physician D speaks bluntly, while others place particular importance on the way the physician D treats patients. Furthermore, some patients D do not want to see a physician D unless the physician D has graduated from a certain university, and some patients D definitely do not want to see a physician D unless the physician D is a woman. Alternatively, there may be someone who is willing to see a physician D if the physician D is in his thirties, has a gentle personality, and is a good-looking man who graduated from a certain university.

Through the present services, a patient U can search for a medical institution or a physician D matching the demands of the patient U because a search can be performed based on one or more factors selected by the patient U from such a plurality of factors.

The information presented to the patient U as a result of the search includes information (e.g., an ID) that uniquely identifies a medical institution or a physician D that matches or has a high degree of matching the wishes of the patient U, as well as the content of evaluations of the medical institution or the physician D by other patients U. Through the above, the patient U using the search service can specifically decide on a medical institution or a physician D with whom to make an appointment by referring to the content of various evaluations by other patients U or physician information described later.

The services for physicians or the like are services which support physicians D giving medical care to patients U and medical students who are future physicians D. For a specific example, the services for physicians or the like include a physician registration service and a search service.

The physician registration service among the services for physicians or the like is a service which supports physicians D and students who are trying to use the present services in registering for the present services.

The physician registration service allows a physician D or a student to use dedicated application software (referred to in the following as a “physician app”) installed to a physician terminal 3. The physician D or the student can use the physician app to easily register for the present services by inputting information (referred to in the following as “physician registration information”) necessary to register for the present services.

Here, the physician registration information is not particularly limited, but may include a captured image, name, date of birth, place of birth, place of residence, school (e.g., university, high school, junior high school, and elementary school), club membership, hobbies, marital status, family structure, occupation and final educational background of relatives (e.g., parents and siblings), and a social networking service (SNS) account of the physician D. The registration information can also include subjects in which the physician D specializes. For a specific example, when the physician D is a dentist, specialties may include implantation, orthodontia, whitening, root canal treatment, dentures, and aesthetic dentistry.

Note that because the physician registration information is utilized as a search target (previously described factor) of the search service used by a patient U, more detailed information is preferable. The more detailed the registration information, the more useful the registration information is in determining the personality of the physician D. Therefore, the search service for the patient U can be enhanced.

In the registration service, items such as a captured image, name, address, telephone number, access method, introduction, characteristics, and medical care content of a medical institution can also be registered as information (referred to in the following as “medical institution information”) about the medical institution to which the physician D belongs. Note that the medical institution information is stored and managed in a medical institution DB 182 of the server 1.

The physician D using the registration service can also refer to evaluations of the physician D or the medical institution to which the physician D belongs from patients U. For a specific example, the physician D can refer to evaluations from patients U for all previously performed medical care.

Through the above, even when receiving a poor evaluation, the physician D can refer to the evaluation and attempt to increase their skill by improving points needing improvement. When obtaining a good evaluation by contrast, the physician D can take encouragement for their medical care.

The physician D using the registration service can also register their own medical care history in the present services as medical care history information separately from the physician registration information.

The medical care history information includes items such as specific treatment content by the physician D for a patient U.

Because the medical care history information is utilized as a search target (various factors used for search) of the search service used by the patient U in addition to the physician registration information, more detailed information is preferable. The more detailed the medical care history information, the more useful the medical care history information is in determining the skill (ability) of the physician D. Therefore, the search service for the patient U can be enhanced.

Note that the physician registration information and medical care history information of the physician D, which are registered by the physician D using the present services, are both stored and managed in the physician DB 181 of the server 1 as information included in information (referred to in the following as “physician information”) about the physician D.

The registration service may also include an SNS function.

Through the above, the physician D or the student using the present services can upload daily events as a diary or comments along with videos, pictures, or the like. Furthermore, the physician D or the student can add other physicians D or students using the present services as “friends”.

As a result, the physician D can make horizontal connections by joining a circle, for example. The student can also make horizontal connections from an early stage before becoming a physician D by joining circles and associations in which physicians D also participate, and by adding other students and physicians D who use the present services as “friends”.

The search service among the services for physicians or the like is a service which supports a physician D in searching for workshops or managing materials used to produce workpieces (referred to in the following simply as “materials”).

Through the search service, the physician D uses the physician app to input items such as an area covered by a workshop which performs tasks such as production of a workpiece, and the number of workpieces the physician D wants to request for production. Through the above, the physician D can easily search for a workshop that matches or has a high degree of matching the wishes of the physician D.

The information presented to the physician D as a result of the search includes information (e.g., an ID) that uniquely identifies a workshop that matches or has a high degree of matching the wishes of the physician D, as well as the content of evaluations of the workshop by other physicians D. Through the above, the physician D using the search service can refer to the content of various evaluations by other physicians D to specifically decide on a workshop from which a workpiece is to be ordered.

According to the search service, the physician D also uses the physician app to input items such as a category or an amount of a material. Through the above, the physician D can easily search for a person who sells materials (referred to in the following as a “material seller”) that matches or has a high degree of matching the wishes of the physician D.

The information presented to the physician D as a result of the search includes information (e.g., an ID) that uniquely identifies the material seller that matches or has a high degree of matching the wishes of the physician D, as well as the content of evaluations of the material seller by other physicians D. Through the above, the physician D using the search service can refer to the content of various evaluations by other physicians D to specifically decide on a material seller from which material is to be ordered.

The physician D using the search service can also establish a regular order from a material seller. For a specific example, a minimum storage quantity of a material can be set in advance so that an automatic order is placed before the stored amount of the material falls below the minimum storage quantity.

The services for workshops are services which support workshops. A workshop receives a production order for a workpiece from a physician D or a medical institution. The services for workshops allow the workshop to use dedicated application software (referred to in the following as a “workshop app”) installed to a workshop terminal 4 to confirm information about the physician D or the medical institution that placed the order. Specifically, the workshop can use the workshop app to disclose congestion status of the workshop by registering information (referred to in the following as “workshop information”) indicating items such as the order status or work status of the workshop. Through the above, orders from physicians D can be efficiently received. Note that the registered workshop information is stored and managed in a workshop DB 185.

Services for material sellers are services which support material sellers. The workshop receives an order for material from a physician D or a medical institution. The services for material sellers allow a material seller to use dedicated application software (referred to in the following as a “material seller app”) installed to a material seller terminal to confirm information about the physician D or the medical institution that placed the order. Specifically, the material seller can use the material seller app to automatically receive an order by registering information (referred to in the following as “material information”) about the material beforehand. Note that the registered material information is stored and managed in a material DB 186.

In this manner, the present services enable support of patients U, physicians D, medical institutions, workshops, and material sellers, thus allowing the patients U, the physicians D, the medical institutions, the workshops, and the material sellers to enjoy various advantages.

To summarize the above, the present services allow patients U to enjoy the following advantages. That is, a patient U can easily search for a nearby medical institution or physician D and easily confirm availability information. The patient U can make an appointment using the patient terminal 2 as a patient registration card. The patient U can confirm the medical care history of the patient U and evaluate the medical care of the patient U. The patient U is given incentives such as points that are specific to the present services or that can be used with other services. The patient U can also exchange the given points for items such as benefits at medical institutions.

The present services also allow physicians D or medical institutions to enjoy the following advantages. That is, a physician D or a medical institution can perform searches or referrals. The physician D or the medical institution can easily manage appointments and patients U. The physician D or the medical institution can confirm evaluations of the physician D or the medical institution by patients U. The physician D or the medical institution can offer benefits specific to the medical institution and can attempt to differentiate themselves from other medical institutions.

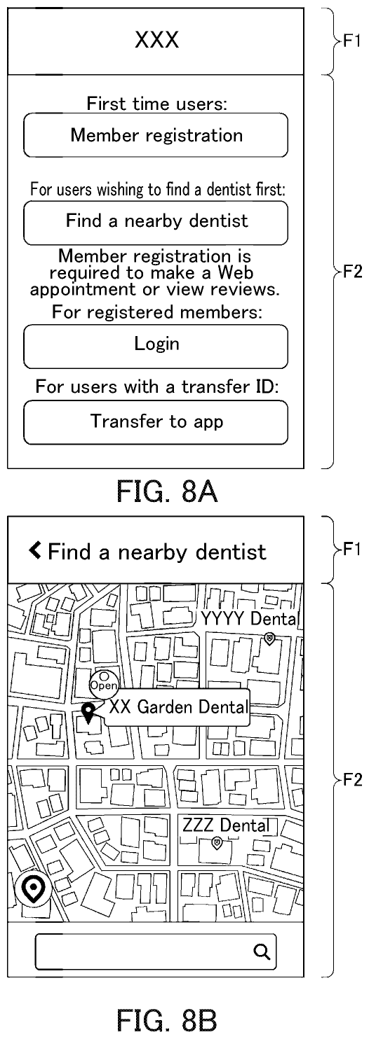

FIGS. 8A to 11B are diagrams illustrating specific examples of images displayed by a patient terminal of a patient using the present services.

In FIG. 8A, an example of a case in which a patient U is receiving dental treatment at a dental clinic is illustrated as an example of a top screen displayed by the patient terminal 2. As illustrated in FIG. 8A, the patient terminal 2 includes display regions F1 and F2. The display region F1 displays items such as the name of the present service or the name of the patient app. The display region F2 displays a button labeled “Member registration”, a button labeled “Find a nearby dentist”, a button labeled “Login”, and a button labeled “Transfer to app”.

When the button labeled “Member registration” is pressed, the screen transitions to a screen (not illustrated) for inputting patient information to register for the present services. The button labeled “Member registration” is a button pressed by a patient U using the patient app for the first time.

When the button labeled “Find a nearby dentist” is pressed, the screen transitions to a screen for finding a dental clinic as illustrated in FIG. 8B. The button labeled “Find a nearby dentist” is a button for displaying on a map the locations of dental clinics near the present location of the patient U. Note that the button labeled “Find a nearby dentist” can be pressed to display a map before member registration is performed, but member registration is required to make an appointment at a dental clinic displayed on the map or view reviews (evaluations).

Also note that the search method “Find a nearby dentist” is optional and not a limitation. The provider of the present services may employ various search methods including, for example, “Search by present location”, “Search by prefecture”, “Search by station name”, “Search by service content”, “Search by keyword”, and “Search by XX equipment”.

When the button labeled “Login” is pressed, the screen transitions to an input screen (not illustrated) for logging into the present services. The button labeled “Login” is to be pressed by a patient U who has already registered as a member.

When the button labeled “Transfer to app” is pressed, the screen transitions to a screen (not illustrated) on which a procedure (referred to in the following as a “app transfer procedure”) is performed to allow a patient U, who has thus far received medical care using a patient registration card made of paper or the like, to make a medical care appointment using the patient app. The screen on which the app transfer procedure is performed displays items such as information (e.g., an ID) that uniquely identifies a dental clinic assigned to the dental clinic by the present services, a patient registration number shown on the patient registration card made of paper or the like, and information (e.g., a transfer ID) uniquely identifying the patient U associated with the patient information of the patient U performing the app transfer procedure. The button labeled “Transfer to app” is a button pressed by a patient U who already has information (e.g., a transfer ID) uniquely identifying the patient U.

FIG. 8B illustrates an example of a screen displayed by the patient terminal 2 when the button labeled “Search for a nearby dentist” is pressed in the top screen displayed by the patient terminal 2.

As illustrated in FIG. 8B, the title “Find a nearby dentist” is displayed in the display region F1. The display region F2 displays items such as icons on a map indicating dental clinics near the present location of the patient U. The patient U can designate a dental clinic by pressing an icon or the like displayed on the map indicating a dental clinic and easily confirm information about the dental clinic. In addition, the patient U can easily make an appointment for medical care as needed.

Note that among the dental clinics displayed on the map by icons or the like, a dental clinic that has been designated by pressing of the patient U is referred to in the following as a “designated dental clinic”.

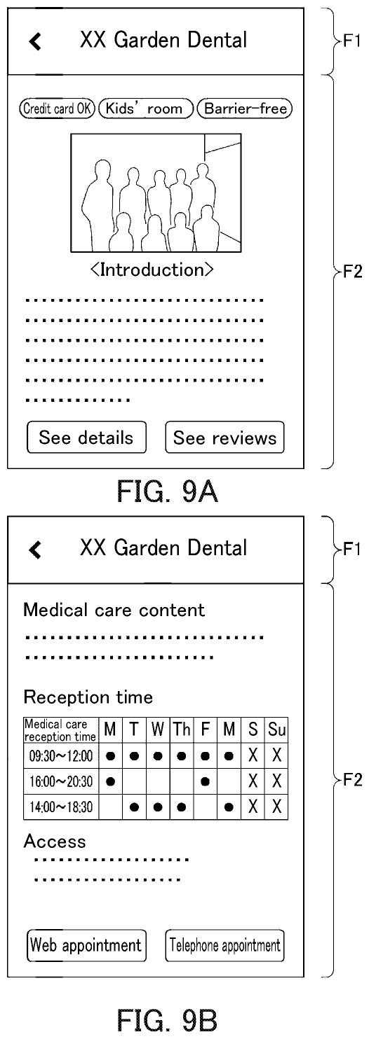

FIG. 9A illustrates an example of a screen displayed by the patient terminal 2 when a dental clinic displayed on the map as an icon or the like has been pressed.

As illustrated in FIG. 9A, items such as a specific name of the designated dental clinic are displayed in the display region F1. The display region F2 displays a captured image of the staff of the designated dental clinic, an introduction to the designated dental clinic, a button labeled “See details”, and a button labeled “See reviews”.

The patient U reads the introduction to the designated dental clinic displayed in the display region F2, and when the patient U wants to see more detailed information, the patient U presses the button labeled “See details”. When the button labeled “See details” is pressed, medical institution information of the designated dental clinic is displayed as illustrated in FIG. 9B.

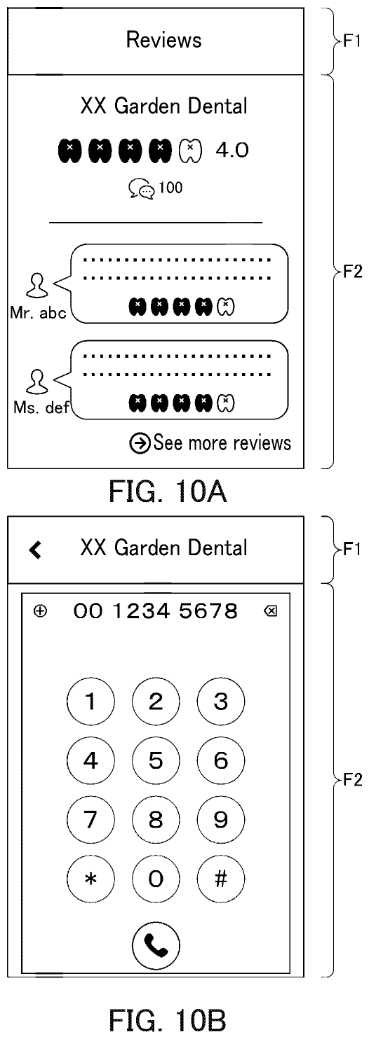

Furthermore, when the patient U wants to see reviews (evaluations) of the designated dental clinic, the patient U presses the button labeled “See reviews”. When the button labeled “See reviews” is pressed, evaluation information (reviews) of the designated dental clinic is displayed as illustrated in FIG. 10A. Specifically, the title “Reviews” is displayed in the display region F1. The display region F2 displays the results of five-point evaluations, the total number of reviews, and the content of each evaluation by a patient U (review and five-point evaluation) of the designated dental clinic by patients U.

Returning to FIG. 9B, FIG. 9B illustrates an example of a screen displayed by the patient terminal 2 when the button labeled “See details” displayed in the display region F2 in FIG. 9A is pressed.

As illustrated in FIG. 9B, items such as a specific name of the designated dental clinic are displayed in the display region F1. The display region F2 displays medical institution information of the designated dental clinic. As an example of the medical institution information of the designated dental clinic, FIG. 9B illustrates medical care content, reception time, access method, a button labeled “Web appointment”, and a button labeled “Telephone appointment”.

The patient U reads the medical institution information of the designated dental clinic displayed in the display region F2, and when the patient wants to make an appointment by Web, the patient U presses the button labeled “Web appointment”. When the button labeled “Web appointment” is pressed, an input screen (not illustrated) for a medical questionnaire is displayed. When the patient U finishes inputting the medical questionnaire, a screen for confirming and deciding on the content of the appointment is displayed and the appointment is completed by pressing a button (not illustrated) indicating appointment decision.

Here, when making a return visit rather than a first visit, the patient U can evaluate previous medical care in addition to making an appointment.

The patient U reads the medical institution information of the designated dental clinic displayed in the display region F2, and when the patient U wants to make an appointment by telephone, the patient U presses the button labeled “Telephone appointment”. When the button labeled “Telephone appointment” is pressed, a dial screen illustrated in FIG. 10B is displayed. The display region F1 of the dial screen illustrated in FIG. 10B displays the name of the designated dental clinic. The display region F2 displays items such as a telephone number of the designated dental clinic and a call button. Through the above, the patient U can call the designated dental clinic directly to make an appointment.

In the above example, the patient U referred to the evaluation information (reviews) of the designated dental clinic as illustrated in FIG. 10A when determining things such as whether or not to make an appointment for medical care.

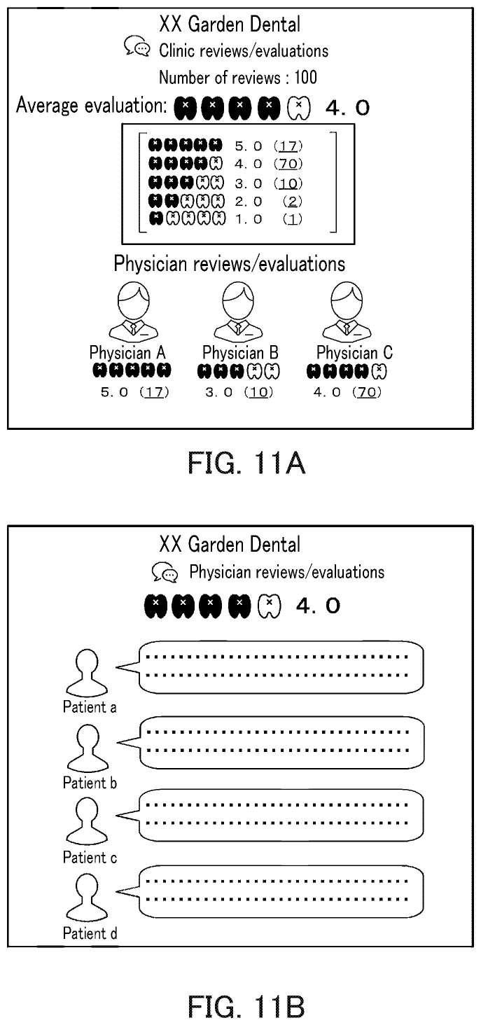

However, some patients U may request an appointment for medical care from a particular physician D rather than from a medical institution in general. To meet such a request, the patient terminal 2 can also display a screen such as that illustrated in FIG. 11A or 11B.

The screen illustrated in FIG. 11A displays the result of five-point evaluations (average evaluation result), the total number of reviews, and the results of each five-point evaluation by a patient U (each evaluation result) of the designated dental clinic (XX Garden Dental) as evaluation information of the designated dental clinic.

In addition, the screen illustrated in FIG. 11A displays the result of five-point evaluations (average evaluation result) and a total number of reviews for each physician D (physicians A to C in FIG. 11A) belonging to the designated dental clinic (XX Garden Dental).

In FIG. 11B, the actual content of reviews posted by each patient U (patients a to d in the example of FIG. 11B) is displayed.

In this manner, a patient U can easily confirm items such as the results of evaluations and the content of reviews not only for a medical institution in general but for individual physicians D. As a result, the patient U can receive (or make an appointment for) medical care that is more favorable to the patient U.

In addition, the patient U can perform tasks such as various kinds of search by setting the object of medical care (or an appointment therefor) with a physician D as the main target rather than a medical institution.

For example, the patient U can perform such tasks as various kinds of search based on a ranking of physicians D based on evaluations of the physicians D from various kinds of viewpoints.

For a specific example, the patient U can decide on a desired physician D from whom to receive medical care (or make an appointment therefor) based on a ranking of physicians D present within 10 km of the residence of the patient U.

Next, a configuration of an information processing system which implements the provision of the present services described above, that is, an information processing system according to an embodiment of the present invention, is described using FIG. 12.

FIG. 12 is a diagram illustrating the configuration of the information processing system according to the first embodiment of the present invention.

The information processing system illustrated in FIG. 12 is configured to include the server 1, patient terminals 2-1 to 2-n, physician terminals 3-1 to 3-n, the workshop terminal 4, and the seller terminal 5.

The server 1, the patient terminals 2-1 to 2-n, the physician terminals 3-1 to 3-m, the workshop terminal 4, and the seller terminal 5 are connected to each other through a predetermined network N such as the Internet.

The server 1 is managed by the service provider G and performs various processing to implement the present services while communicating with the patient terminals 2-1 to 2-n, the physician terminals 3-1 to 3-m, the workshop terminal 4, and the seller terminal 5 as appropriate.

The patient terminals 2-1 to 2-n are information processing devices operated by respective patients U1 to Un and are configured as devices such as personal computers, smartphones, or tablet computers. Note that the patient terminals 2-1 to 2-n are collectively referred to as a “patient terminal 2” and the patients U1 to Un are collectively referred to as a “patient U” when there is no need to distinguish between them.

The physician terminals 3-1 to 3-m are information processing devices operated by respective physicians D1 to Dm and are configured as devices such as personal computers, smartphones, or tablet computers. Note that the physician terminals 3-1 to 3-m are collectively referred to as a “physician terminal 3” and the physicians D1 to Dm are collectively referred to as a “physician D” when there is no need to distinguish between them.

The workshop terminal 4 is an information processing device operated in a workshop and is configured as a device such as a personal computer, a smartphone, or a tablet computer.

The seller terminal 5 is an information processing device operated by a material seller and is configured as a device such as a personal computer, a smartphone, or a tablet computer.

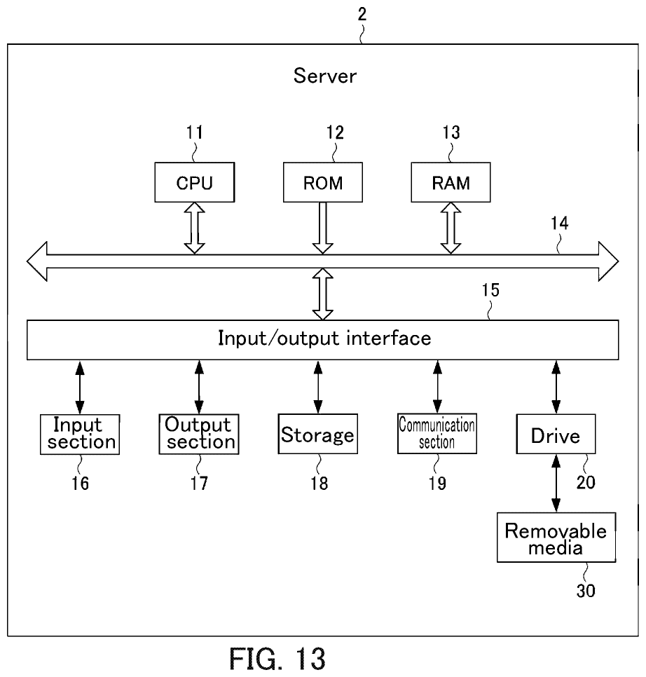

FIG. 13 is a block diagram illustrating an example of a hardware configuration of the server in the information processing system illustrated in FIG. 12.

The server 1 includes a central processing unit (CPU) 11, read-only memory (ROM) 12, random-access memory (RAM) 13, a bus 14, an input/output interface 15, an input section 16, an output section 17, storage 18, a communication section 19, and a drive 20.

The CPU 11 executes various processing according to a program stored in the ROM 12 or a program loaded into the RAM 13 from the storage 18.

Items such as data necessary for the CPU 11 to execute the various processing are appropriately stored in the RAM 13.

The CPU 11, the ROM 12, and the RAM 13 are connected to each other through the bus 14. The input/output interface 15 is also connected to the bus 14. The input section 16, the output section 17, the storage 18, the communication section 19, and the drive 20 are connected to the input/output interface 15.

The input section 16 is configured as a keyboard, for example, and outputs various information.

The output section 17 is configured as a speaker or a display such as a liquid-crystal display, and outputs various information as images or sound.

The storage 18 is configured as dynamic random-access memory (DRAM) or the like, and stores various data.

The communication section 19 performs communication between the other devices (e.g., the patient terminal 2, the physician terminal 3, the workshop terminal 4, and the seller terminal 5 in FIG. 12) through the network N including the Internet.

Removable media 30 such as a magnetic disk, an optical disk, a magneto-optical disk, or semiconductor memory is appropriately mounted in the drive 20. A program read from the removable media 30 by the drive 20 is installed to the storage 18 as necessary.

The removable media 30 can also store the various data stored in the storage 18 in the same manner as the storage 18.

Although not illustrated in the drawings, the patient terminal 12, the physician terminal 3, the workshop terminal 4, and the seller terminal 5 in FIG. 12 can also have the same basic configuration as the hardware configuration illustrated in FIG. 13.

Accordingly, description of the configuration of the patient terminal 2, the physician terminal 3, the workshop terminal 4, and the seller terminal 5 is omitted.

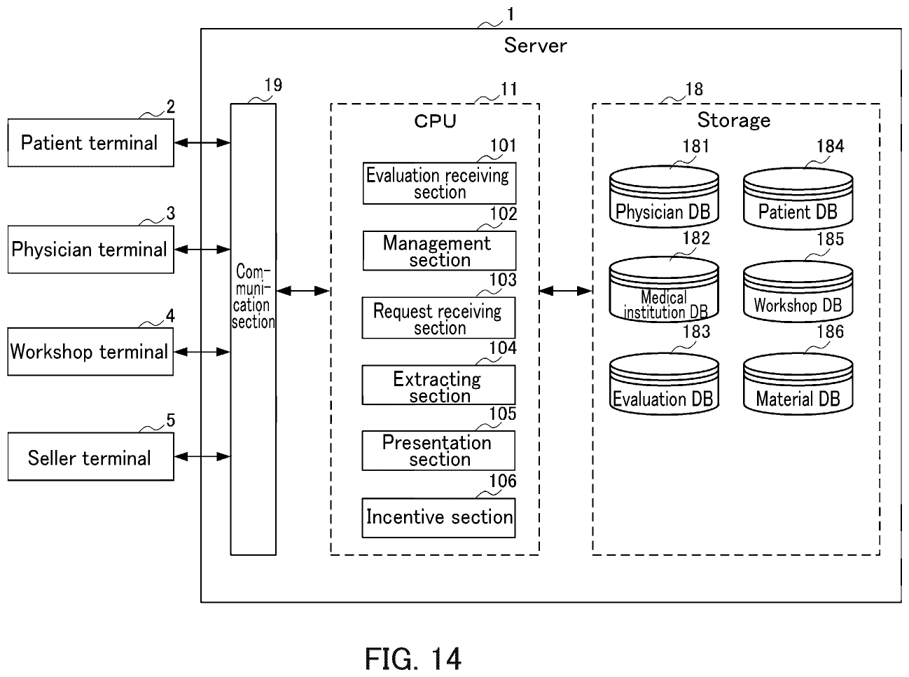

Next, a functional configuration of the server 1 having such a hardware configuration is described with reference to FIG. 14.

FIG. 14 is a functional block diagram illustrating an example of a functional configuration for implementing evaluation support processing within the functional configuration of the server 1 in FIG. 13 in the information processing system in FIG. 12.

“Evaluation support processing” refers to processing to support evaluation of a physician D or a medical institution by a patient U in the present services described above.

When the evaluation support processing is executed as illustrated in FIG. 14, the CPU 11 (FIG. 13) of the server 1 functions as an evaluation receiving section 101, a management section 102, a request receiving section 103, an extracting section 104, a presentation section 105, and an incentive section 106.

Note that the physician DB 181, the physician DB 182, the evaluation DB 183, the patient DB 184, the workshop DB 185, and the material DB 186 are arranged in one region of the storage 18 (FIG. 13).

The evaluation receiving section 101 receives an evaluation of a physician D by a patient U.

Specifically, when one or more patients U who have received medical care from a physician D each operate the patient terminal 2 to evaluate the physician D, the evaluation receiving section 101 receives the evaluations.

Furthermore, when one or more physicians D who have ordered workpieces from a workshop each operate the physician terminal 3 to evaluate the workshop, the evaluation receiving section 101 receives the evaluations.

When one or more physicians D who have ordered materials from a material seller each operate the physician terminal 3 to evaluate the material seller, the evaluation receiving section 101 receives the evaluations.

The management section 102 manages, for each of n (n being an integer value of 1 or greater) physicians D, the physician information of the physician D including at least the evaluation information of the physician D by the patients U received by the evaluation receiving section 101.

Specifically, the management section 102 stores and manages the physician information for each of the n physicians D in the physician DB 181. The management section 102 also stores and manages evaluations of each of the n physicians D by each of one or more patients U in the evaluation DB 183 as evaluation information for each of the n physicians D.

The management section 102 also stores and manages the workshop information registered through the workshop terminal 4 by each of one or more workshops in the workshop DB 185. The management section 102 also stores and manages evaluations of each of the one or more workshops by each of one or more physicians D in the evaluation DB 183 as evaluation information for each of the one or more workshops.

The management section 102 also stores and manages the material information registered through the seller terminal 5 by each of the one or more material sellers in the material DB 186. The management section 102 also stores and manages evaluations of each of the one or more material sellers by each of one or more physicians U in the evaluation DB 183 as evaluation information for each of the one or more material sellers.

The request receiving section 103 receives a request from a patient U seeking medical care from a desired physician D.

Specifically, when the patient U inputs a request for a physician D from whom the patient U seeks medical care by operating a patient terminal 2, the request receiving section 103 receives information (referred to in the following as “patient request information”) based on the input content.

The extracting section 104 extracts m (m being an integer value no greater than n and no less than 0) physicians D to be presented to the patient U among the n physicians D managed by the management section 102 based on at least part of the physician information stored and managed in the physician DB 181 by the management section 102 for each of the n physicians D and the patient request information received by the request receiving section 103.

The presentation section 105 presents information to the patient U based on the physician information of each of the m extracted physicians D.

Specifically, the presentation section 105 presents information to the patient U based on the physician information of each of the m physicians D extracted from the physician DB 181 by the extracting section 104.

Here, “information based on the physician information” includes items such as all the physician information of the physicians D, a portion of the physician information, and information newly generated or processed based on the physician information.

Note that when the number of extracted physicians D is 0 (i.e., no physicians D are extracted), the patient terminal 2 displays a message stating “0 results” or a message stating “no one applicable”, for example.

When an evaluation is received by the evaluation receiving section 101, the incentive section 106 gives a predetermined reward to the patient U giving the evaluation according to the content of the evaluation.

For a specific example, the incentive section 106 may give more incentives (e.g., points) when the content of the evaluation of a physician D by the patient U is a comment (review) than when the content is a multiple-point evaluation (e.g., five-point evaluation). The incentive section 106 may also give more incentives (e.g., points) than usual when the comment (review) exceeds a preset predetermined number of characters.

Through the above, the patient U can be expected to actively try to evaluate physicians D.

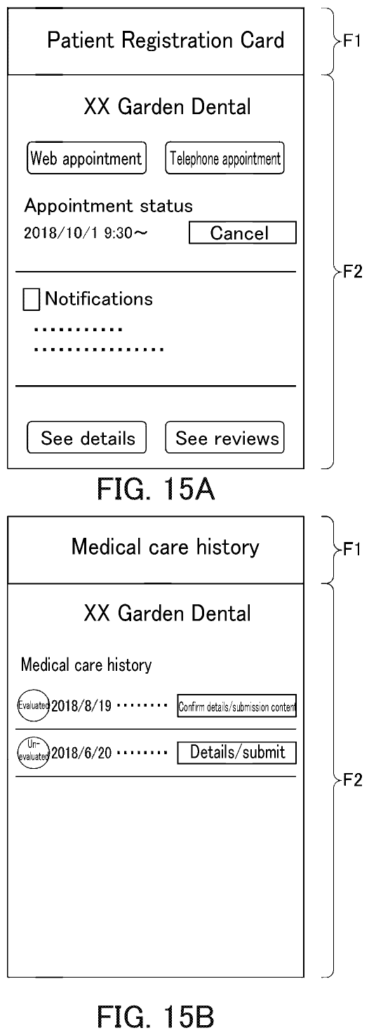

Next, a specific example of the present services is described with reference to FIGS. 15 to 20.

FIG. 15A illustrates a specific example of a patient terminal 2 being used as a patient registration card of a designated dental clinic.

As described above, when making an appointment at a designated dental clinic, a patient U can make an appointment by Web or by telephone by operating the patient terminal 2. In so doing, the patient U can use the patient terminal 2 as a patient registration card.

In this case, the display region F1 displays the title “Patient Registration Card” as illustrated in FIG. 15A. The display region F2 displays the name of the designated dental clinic, a button labeled “Web appointment”, a button labeled “Telephone appointment”, the current appointment status of the patient U, a button labeled “Cancel”, a notice from the designated dental clinic, a button labeled “Medical history”, and a button labeled “Coupon”.

The screen transitions of the patient terminal 2 when the button labeled “Web appointment” is pressed and when the button labeled “Telephone appointment” is pressed are the same as in the case of FIG. 9B described above, and description thereof is therefore omitted.

The current appointment of the patient U can be canceled by pressing the button labeled “Cancel”. For a specific example, as illustrated in FIG. 15A, when an appointment has been made with “2018/10/1 9:30˜” as an “appointment status”, the patient U can cancel the appointment by pressing the button labeled “Cancel”.

When the button labeled “Medical care history” is pressed, a history of past medical care of the patient U is displayed. Specifically, when the button labeled “Medical care history” is pressed, the screen illustrated in FIG. 15B is displayed. Herein, the display region F1 displays the title “Medical care history”. The display region F2 displays the name of the designated dental clinic and each item of medical care history. Each item of the medical care history displayed in the display region F2 is given an icon labeled “evaluated” or “unevaluated”. Herein, items of the medical care history given the icon labeled “evaluated” are items of the medical care history that have already been evaluated by the patient U. By contrast, items of the medical care history given the icon labeled “unevaluated” are items of the medical care history that have not been evaluated by the patient U.

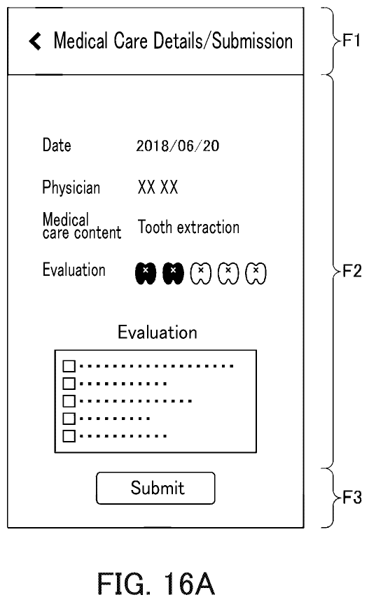

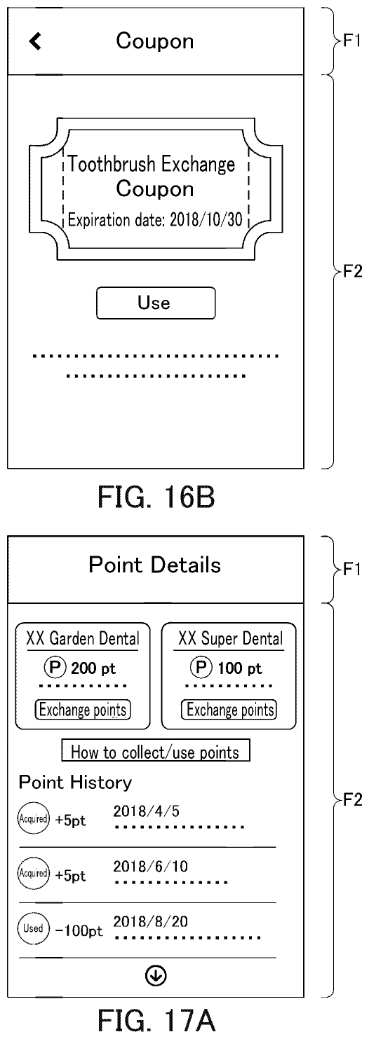

Furthermore, a button labeled “Confirm details/submission content” is displayed on each item of the medical care history given the icon labeled “Evaluated”. Detailed information of the medical care history and specific content of an evaluation previously given by the patient U can be confirmed by pressing the button labeled “Confirm details/submission content”. Furthermore, a button labeled “Details/submit” is displayed on each item of the medical care history given the icon labeled “Unevaluated”. Detailed information of the medical care history can be confirmed, and the patient U can evaluate the medical care by pressing the button labeled “Details/submit”. Specifically, when the button labeled “Details/submit” is pressed, the screen illustrated in FIG. 16A is displayed by the patient terminal 2.

Returning to FIG. 15A, when the button labeled “Coupon” displayed in the display region F is pressed, information about a coupon (referred to in the following as “coupon information”) that the patient U can enjoy is displayed by the patient terminal 2. Specifically, when the button labeled “Coupon” is pressed, the screen illustrated in FIG. 16B is displayed by the patient terminal 2.

Note that the method of obtaining a patient examination card number illustrated in FIG. 15A is an example and not a limitation. The patient registration card number may be obtained by any method. For example, the patient registration card number may be obtained by any method such as a bar code or a QR CODE (registered Japanese trademark).

FIG. 16A illustrates a specific example of a screen displayed by the patient terminal 2 when the button labeled “Details/submit” in the screen in FIG. 15B is pressed.

The screen illustrated in FIG. 16A is configured to include display regions F1 to F3. Herein, the display region F1 displays the title “Medical Care Details/Submission”. The display region F2 displays a date on which medical care was performed (2018/06/20), the name of the physician D who administered the medical care (XX XX), the content of the medical care (tooth extraction), an entry field for a five-point evaluation of the medical care by the patient U, and a check box input field for evaluation content. Note that in the example in FIG. 16A, the five-point evaluation of the medical care by the patient U is “2”. Furthermore, in the check box input field of the evaluation content, check box items such as “I felt that the treatment was painful or inadequate”, “The explanation was insufficient”, “The customer service attitude was not good”, “I had to wait longer than necessary”, and “They did not listen to me” can be displayed. The patient U can input checks for any of these check box items that are applicable.

The display region F3 in FIG. 16A displays a button labeled “Submit”. The patient U can register an evaluation of medical care by pressing the button labeled “Submit” after completing input into a field for input of the evaluation of the medical care displayed in the display region F2.

FIG. 16B illustrates a specific example of a screen displayed by the patient terminal 2 when the button labeled “Coupon” in the screen in FIG. 15A is pressed.

The screen illustrated in FIG. 16B is configured to include display regions F1 and F2. Herein, the display region F1 displays the title “Coupon”. The display region F2 displays a coupon labeled “Toothbrush Exchange Coupon Expiration date: 2018/10/30” as a coupon that the patient U can enjoy. The patient U can receive a toothbrush as a present by using the coupon at an applicable dental clinic within the period of validity. Note that when using the coupon, the patient U must press the button labeled “Use” displayed in the display region F2. That is, as illustrated in FIG. 16B, the coupon is put into a used state by pressing the button labeled “Use”, and the coupon can actually be used by presenting the coupon to the applicable dental clinic in the used state.

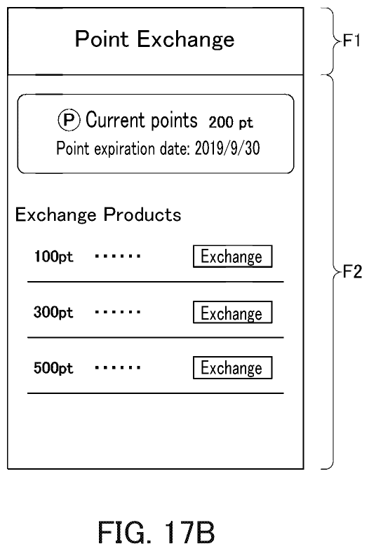

FIG. 17A illustrates a specific example of points given to the patient U as an incentive.

The screen illustrated in FIG. 17A is configured to include display regions F1 and F2. Herein, the display region F1 displays the title “Point Details”. The display region F2 displays information about the points of the patient U.

Specifically, the display region F2 displays information (referred to in the following as “coupon exchange information”) about a dental clinic where the points can be exchanged for a coupon. Specifically, coupon exchange information “XX Garden Dental P 200 pt Expiration date: 2019/09/30” and coupon exchange information “XX Super Dental P 100 pt Expiration date: 2019/08/31” are displayed by icons. Each icon displays a button labeled “Exchange points”. The patient U can exchange the points for a coupon by pressing the button labeled “Exchange points”.

The display region F2 also displays a button labeled “How to collect/use points”. The patient U can obtain information about how to acquire and collect points efficiently or how to use points well by pressing the button labeled “How to collect/use points”.

The display region F2 also displays points that the patient U has acquired thus far and a history of points used. Specifically, icons indicating the acquisition or use of points, the number of points acquired or used, the date on which the points were acquired or used, and the reason for acquiring or using the points are displayed in chronological order for each item of history of acquiring or using points. Note that although the history of acquiring or using points illustrated in FIG. 17A only displays three cases, the history of acquisition or usage of points can be viewed sequentially by pressing an icon labeled with a downward arrow displayed at the bottom edge of the display region F2.

FIG. 17B illustrates a specific example of a screen displayed by the patient terminal 2 when the button labeled “Exchange points” in the screen of FIG. 17A is pressed.

The screen illustrated in FIG. 17B is configured to include display regions F1 and F2. Herein, the display region F1 displays the title “Point Exchange”. The display region F2 indicates the number of points that the patient U currently holds and the expiration date of the points. Specifically, “P Current points 200 pt Point expiration date: 2019/9/30” is displayed. By viewing this display, the patient U can confirm the number of points currently held and the expiration date of the points, and can decide whether or not to exchange the points for coupons (rights to exchange products) set by each dental clinic. The display region F2 displays specific examples of “exchange products” that allow points to be exchanged for products. Specifically, “100 pt Toothbrush”, “300 pt Whitening Half-Price”, and “500 pt Free Whitening” are displayed as exchange products. Herein, when exchanging the points for a “toothbrush”, the patient U presses the button labeled “Exchange” displayed to the right of the label “100 pt Toothbrush”. Through the above, the patient U can obtain a coupon exchangeable for a “toothbrush” by using “100 pt” needed to exchange for the “toothbrush” out of the points (200 pt) that the patient U currently holds.

Note that the form of use of the points currently held by the patient U is not particularly limited to a form in which the points are exchanged for a coupon as indicated in 10(B).

For example, the points currently held by the patient U are points (referred to in the following as “class 1 points”) of a type managed by the service provider of the present services. The present services may employ a form in which the class 1 points are exchanged for points (referred to the following as “class 2 points”) of a different type managed by another party who is not illustrated. In this case, the exchange rate of class 1 points to class 2 points can be arbitrarily set.

For another example, the present services may employ a form in which the class 2 points are converted to class 1 points.

By enabling the mutual exchange of class 1 points and class 2 points in this way, a user of the class 2 points can be given an incentive to become a member of the present services, and the expansion of the scale of the present services can thus be easily achieved.

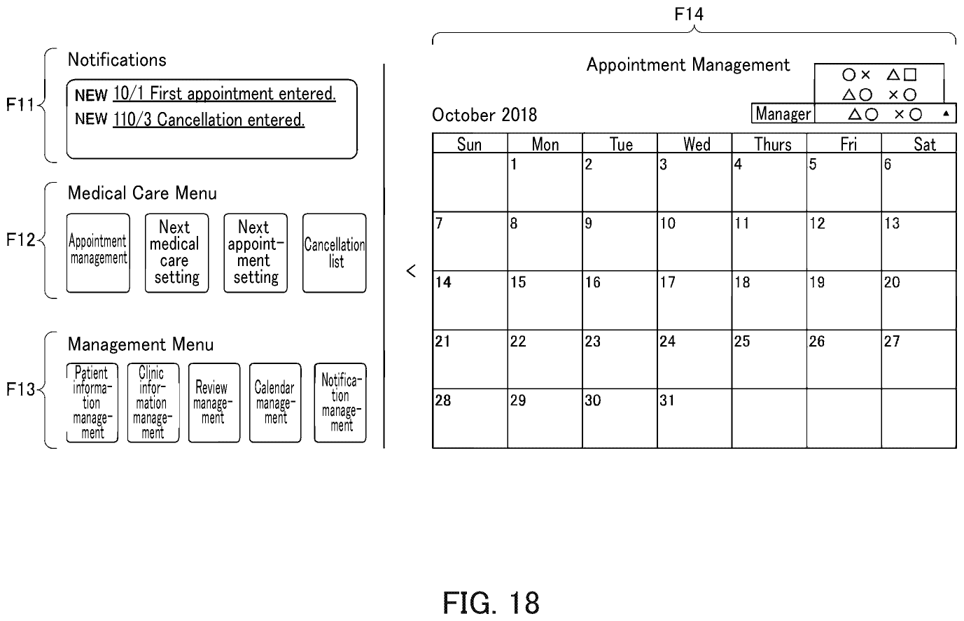

FIG. 18 illustrates a specific example of an appointment management screen displayed by a physician terminal.

The appointment management screen illustrated in FIG. 18 is configured to include display regions F11 to F14.

The display region F11 displays an appointment update status, including newly entered appointments and appointment cancellations, as a “Notice”. Specifically, the display region F11 sequentially displays update statuses such as “NEW 10/1 First appointment entered” and “NEW 10/3 Cancellation entered”. A physician D can confirm progress while viewing the appointment update status displayed in “Notice”.

The display region F12 displays four buttons respectively labeled “Appointment management”, “Next medical care setting”, “Next appointment setting”, and “Cancellation list” as a “Medical Care Menu”. The physician D can easily manage appointments by pressing these buttons.

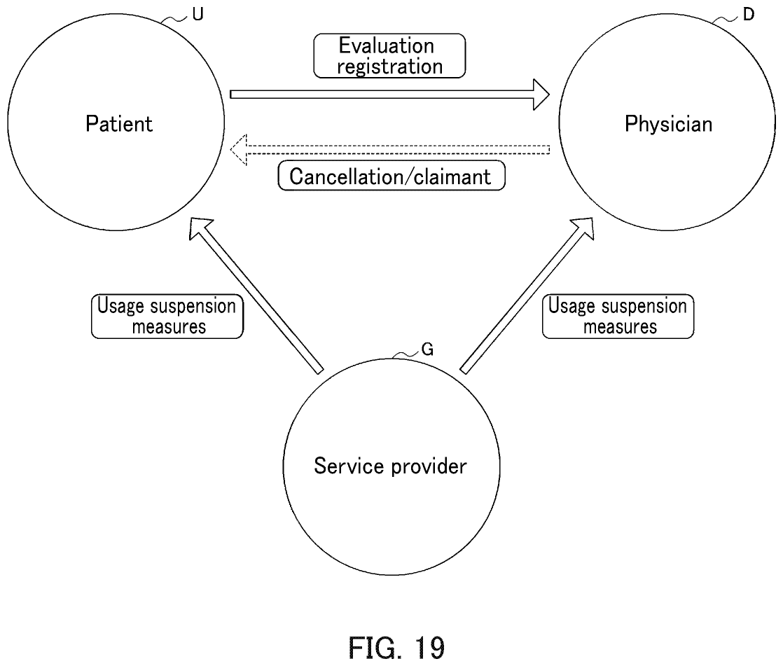

For example, the physician D can perform the operation of making an appointment for medical care by pressing the button labeled “Appointment management”. The physician D can also perform the operation of setting the next medical care content of a patient U by pressing the button labeled “Next medical care setting”. The physician D can also perform the operation of setting the next appointment of a patient U by pressing the button labeled “Next appointment setting”. The physician D can also display a list of canceled appointments by pressing the button labeled “Cancellation list”. By confirming the content displayed in the cancellation list, the physician D can identify a patient U with a large number of no-notice cancellations that may have a negative influence on business. Note that measures to be taken for a patient with a large number of no-notice cancellations are described later with reference to FIG. 19.

The display region F13 displays five buttons respectively labeled “Patient information management”, “Clinic information management”, “Review management”, “Calendar management”, and “Notification management” as a “Management Menu”. The physician D can easily manage appointments by pressing these buttons.

For example, the physician D can perform an operation to manage at least some of the patient information by pressing the button labeled “Patient information management”. The physician D can also perform an operation to manage at least some of the medical institution information by pressing the button labeled “Clinic information management”. The physician D can perform an operation to manage reviews (e.g., comments) in the evaluation information by pressing the button labeled “Review management”. The physician D can also perform an operation to manage medical care times or the like by pressing the button labeled “Calendar management”. The physician D can also perform an operation to manage the content of the notification displayed in the display region F11 by pressing the button labeled “Notification management”.

The display region F14 displays the appointment status of medical care for each physician D on a calendar as “Appointment Management”. Through the above, the physician D can grasp the status of medical care appointments at a glance.

FIG. 19 is an image diagram illustrating an overview of usage suspension measures taken by a service provider.

As described above, patients U using the present services evaluate the medical care of a physician D. Through the above, other patients U can make appointments for medical care by a desired physician D while confirming the content of evaluations of the physician D.

However, some of the patients U evaluating the physician D may be malicious patients U whose evaluations are not justified. By contrast, due to their position in receiving evaluations, the physician D and a medical institution have difficulty in refusing medical care to malicious patients U (referred to in the following as “claimants”) whose evaluations are unjustified.

In such a case, in the present services, measures (referred to in the following as “usage suspension measures”) are taken to prevent a target claimant from using the present services based on a request from the physician D or the medical institution. That is, the service provider G who is in a neutral position takes the usage suspension measures as necessary. Through the above, the occurrence of personal disputes between a patient U and the physician D resulting from evaluation content or lingering grudges can be prevented in advance.

For a specific example, the content of previous evaluations by a patient U considered a claimant are confirmed on the service provider G side, and the usage suspension measures are implemented for the patient U when the content is found to be unjustified.

Furthermore, some patients U who use the present services may make appointments for medical care using the patient app but repeatedly cancel without notice. In such a case, not only will other patients U who could not make appointments be inconvenienced, but the physician D or the medical institution may incur lost profits. As such, in the present services, usage suspension measures are taken for patients U who repeatedly make cancellations without notice based on a request from the physician D or the medical institution. Through the above, inconvenience to other patients U and incursion of lost profits for the physician D or the medical institution due to no-notice cancellations can be prevented.

Note that even when the service provider G takes the usage stoppage measures for a claimant or a patient U who repeatedly makes cancellations without notice, the patient U subject to the usage suspension measures will not be prohibited from using the medical institution but will only be suspended from using the present services.

Furthermore, in the present services, the usage suspension measures described above are taken not only for a patient U but also for a physician D using the present services.

Specifically, the service provider G can take the usage suspension measures for a physician D who receives particularly bad evaluations from patients U. When the usage suspension measures are taken, the physician D using the present services will not be able to enjoy the benefits obtained by using the present services, and can therefore be expected to make efforts to improve evaluations from patients U.

Note that even if the service provider G takes the usage suspension measures for the physician who receives particularly bad evaluations from patients U, the physician D subject to the usage suspension measures will not be unable to perform medical care work but will only be suspended from using the present services.

As described above, the present services allow the service provider C who is in a neutral position, to take the usage suspension measures for patients U and physicians D. Therefore, the quality of the evaluation information and the quality of service by the physicians D can be expected to improve.

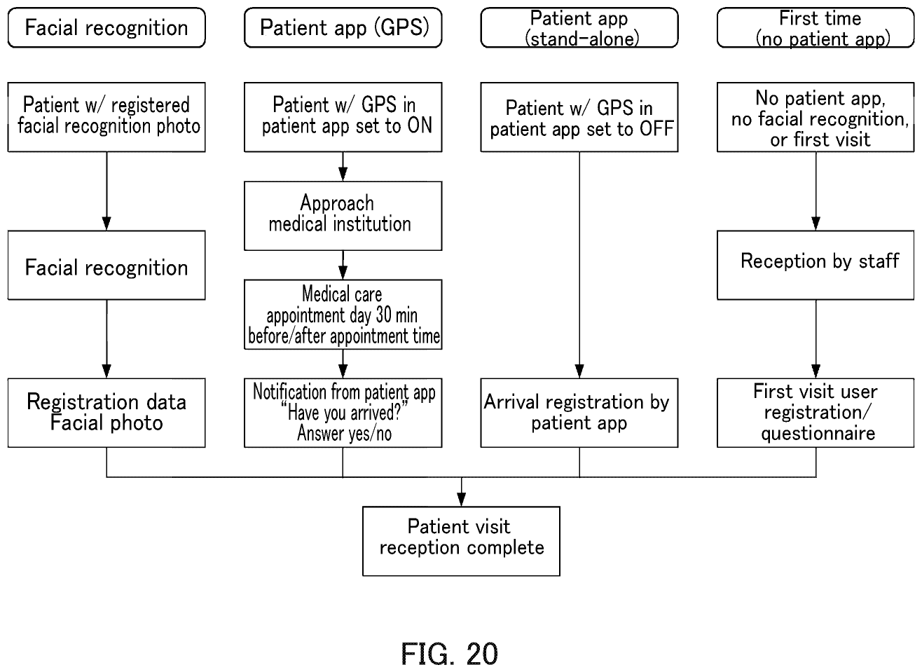

As described above, upon completing a medical care appointment, a patient U visits a medical institution and is received according to the content of the appointment. The reception method in receiving the visit to the medical institution of the patient U using the present services is not particularly limited, but a method such as that illustrated in FIG. 20 may be used for reception, for example.

FIG. 20 is a diagram illustrating a specific example of a reception method when a patient visits the medical institution.

As illustrated in FIG. 20, reception methods when a patient visits the medical institution include reception by “facial recognition”, reception by “patient app (GPS)”, reception by “patient app (stand-alone)”, and reception by “first time (no patient app)”.

When reception is performed by “facial recognition”, data of a captured image of the face of the patient U is registered in advance as patient information. Then, the reception is performed automatically by matching the data of the captured image of the face captured when the patient U visits with the data of the captured image registered in advance.

That is, the method of reception by “facial recognition” is a method used for a patient U who has registered a photograph for facial recognition (i.e., data of a captured image of a face) in advance as patient information as illustrated in FIG. 20.

In a case in which reception is performed by “patient app (location information)”, the reception is automatically performed based on information about the location (e.g., Global Positioning System (GPS) location information) of the device obtained by the patient terminal 2 using entry of a patient U to within a predetermined range of the location of the medical institution as a trigger.

That is, the method of reception by “patient app (position information)” is for example a method used for a patient U whose information about location (e.g., GPS location information) is set to ON (enabled) in the patient app as illustrated in FIG. 20. Specifically, when the patient U approaches the medical institution at which the patient U has made an appointment and has fulfilled predetermined conditions, the patient terminal 2 displays a message such as “Have you arrived?” and buttons for selecting “Yes” or “No”. The patient U operates the patient terminal 2 to perform an input operation to select “Yes” or “No”.

Conditions such as that (1) it is the scheduled day of medical care (day of appointment) and (2) it is within 30 minutes before or after the start time of medical care (time of appointment) can be set as the “predetermined conditions”.

When reception is performed by “patient app (stand-alone)”, the patient U operates the patient terminal 2 to perform reception through the patient app.

That is, the method of reception by “patient app (stand-alone)” is a method of reception used when for example reception by “patient app (location information)” is difficult because a patient U whose information about location (e.g., GPS location information) is turned off (disabled) in the patient app or the patient terminal 2 cannot obtain information about the location of the patient terminal 2 well as illustrated in FIG. 20.

Specifically, when performing reception by “patient app (stand-alone)”, the patient U operates the patient terminal 2 when arriving at the medical institution to register that the patient U has arrived in the patient app. Through the above, reception is performed.

When reception is performed by “first time (no patient app)”, the staff of the medical institution perform reception.

That is, the method of reception by “first time (no patient app)” is a method of reception used in cases where, for example, reception using face recognition or the like cannot be performed because it is the first examination, the patient U does not have a patient terminal 2, or the patient app is not installed to the patient terminal 2 as illustrated in FIG. 20.

Specifically, when performing reception by “first time (no patient app)”, the patient U calls out to the reception staff of the medical institution to perform a conventional reception procedure when arriving at the medical institution. Note that at that time, the staff of the medical institution can inform the patient U that the patient U can enjoy the benefits of using a patient terminal 2 on which the patient app has been installed.

The patient U can be received at the medical institution where the appointment was made by using the above methods.

### Second Embodiment

Next, services for guiding a patient to an examination by a physician at a medical facility where the services in FIGS. 1 to 7 are applied are described.

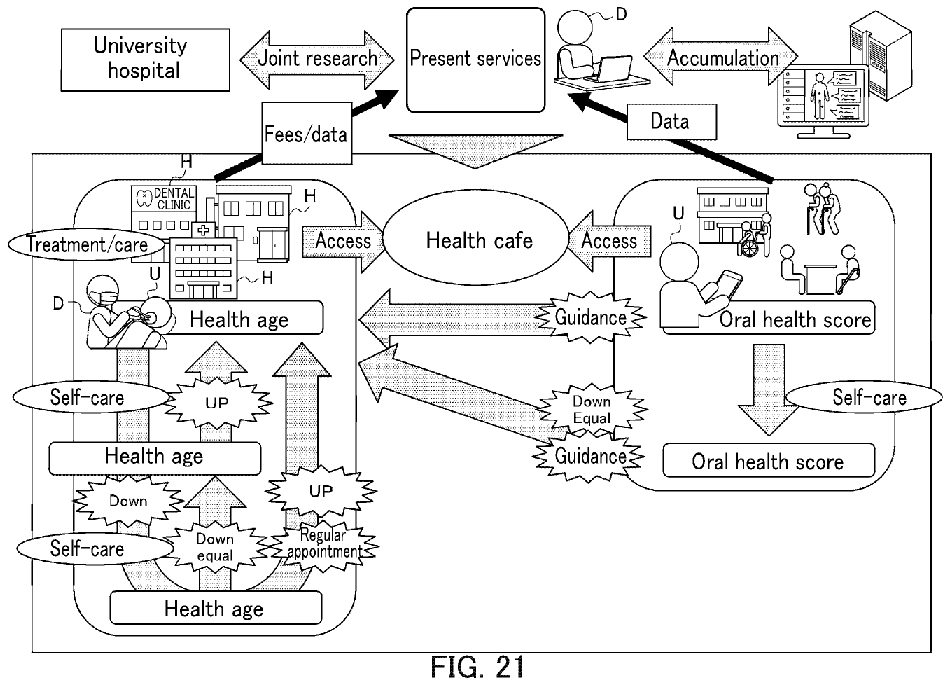

FIG. 21 is a diagram illustrating an overview of an example of the services for guiding the patient to the examination by the physician to whom the services in FIGS. 1 to 7 are applied.

In the services illustrated in FIG. 21, it is assumed that a physician D performs a dental examination of a patient U. In receiving the examination from the physician D, the patient U uses the services illustrated in FIGS. 1 to 7 at a medical facility H (dental clinic).

The physician D performing the dental examination of the patient U indicates an “oral health age” expressing the level of dental health of the patient U in terms of age based on the results of the examination. Basically, oral health age means that the younger the patient is, the higher the level of dental health the patient has (the healthier the patient is). However, what is important in grasping the level of dental health of the patient U is the oral health age of the patient U relative to the actual age of the patient U. This is because, for example, if the oral health age is that of a 30-year-old, it will mean different things if the actual age of the patient U is 20 years old or 40 years old. That is, when the oral health age is less than the actual age of the patient U, the level of dental health can be said to be “high”. When the oral health age is higher than the actual age of the patient U, the level of dental health can be said to be “low”.

A patient U who has a low level of dental health receives an examination from the physician D a second time after performing self-care. When the level indicating dental health is determined to have increased in the second examination, the oral health age indicated by the physician D is younger than in the first examination. For example, when the actual age of the patient U is 20 years old, the oral health age indicated at the end of the first examination is 30 years old, and the oral health age indicated at the start of the second examination is 25 years old, the self-care of the patient U has been effective. In this case, if the patient U continues self-care thereafter, it is possible for the oral health age indicated in the third examination to be lower than the actual age (20 years old) of the patient U.

For another example, when the actual age of the patient U is 20 years old, the oral health age indicated at the end of the first examination is 30 years old, and the oral health age indicated at the start of the second examination is 20 years old, the self-care of the patient U has been fully effective. In this case, because the oral health age of the patient U is the same as the actual age of the patient U, the patient U can increase or maintain their dental health level without having to receive a third examination as long as the patient U continues self-care at that rate. However, after a certain period of time has elapsed from the second evaluation, the present service will perform “guidance” for the patient U with a notification, e-mail, or other means to prompt a third examination. This guidance is an incentive for the patient U to think about receiving a third examination to confirm whether or not self-care is effective.

For another example, when the actual age of the patient U is 20 years old, the oral health age indicated at the end of the first examination is 30 years old, and the oral health age indicated at the start of the second examination is 30 years old, the self-care of the patient U has not been effective. In this case, it is possible that the self-care is inadequate or that the method of self-care is wrong, so the self-care and the like are reconfirmed. The patient U then practices self-care reconfirmed at the second examination in order to obtain a good result (younger oral health age indicated by the physician D) at the third examination.

By accessing a dedicated website (referred to in the following as an “oral health cafe”) provided by the present service, the patient U can confirm the examination history or oral health age history of the patient U at any time. Through the above, the patient U can confirm the effects of self-care on a timeline.

In this manner, self-care to decrease the oral health age may not have a favorable effect if it is not continually performed in a correct manner. As such, the present services provide a method of evaluating the health care of the patient U with an “oral health score” as a method for easy confirmation of whether the patient U is continually practicing self-care in the correct manner. “Oral health score” refers to a predetermined score for each specific item of self-care content. By practicing self-care in the correct manner and reporting (inputting through a dedicated app) as such, the patient U can be given a score corresponding to the self-care. For a specific example, when the oral health score for self-care of “brushing teeth for at least three minutes after breakfast” is “1 point”, the patient U is given that oral health score upon brushing their teeth for at least three minutes after breakfast. For another example, when the oral health score for self-care of “using dental floss to clean between teeth” is “3 points”, the patient U is given that oral health score upon using dental floss to clean between their teeth.

The oral health score given to the patient U is managed by the present service. The patient U can confirm the oral health score given to the patient U at any time by accessing the “oral health cafe” described above.

When the patient U starts self-care after examination by the physician D and the oral health score of the patient U when a predetermined period has elapsed is less than a predetermined score, guidance is performed to recommend that the patient U receive another examination by the physician D. For a specific example, when the oral health score has not reached a predetermined score when two weeks have elapsed after examination, a notification, email, or the like recommending reexamination is send to a smartphone or the like of the patient U as self-care is insufficient.

Through the above, the patient U can receive treatment from the physician D as well as direction on correct self-care by following the guidance (notification or email) from the present service to receive another examination by the physician D. Furthermore, the physician D can increase the number of patients U who actively seek examination in order to be given an oral health score.

Here, the significance of the distinction between “oral health age” and “oral health score” in the present services is as follows. That is, because the “oral health age” can be compared with an actual age in the same manner as a so-called “intestinal age” or “vascular age”, the patient U can get a more realistic sense of the dental health of the patient U. Furthermore, by distinguishing between “oral health age” and “oral health score” so that “oral health age” is only indicated when receiving an examination from the physician D, an increase in the number of people seeking examination from the physician D in order to have their “oral health age” indicated can be expected. By contrast, an “oral health score” can be given by simply performing a predetermined operation through the patient app. In this manner, by making it possible to be easily given an “oral health score” by using the patient app, the patient U can readily use the patient app. As a result, an increase in the number of users of the patient app can be expected.

Data indicating the examination results, an examination fee, and the oral health age indicated by the physician D of the patient U are stored and managed in the present services as “examination data”. Furthermore, data indicating the content of the self-care of the patient U and the oral health score given by the self-care are also stored and managed in the present services as “self-care data”. The examination data and the self-care data managed by the present services is accumulated and utilized for various research (e.g., joint research with university hospitals) or the like.

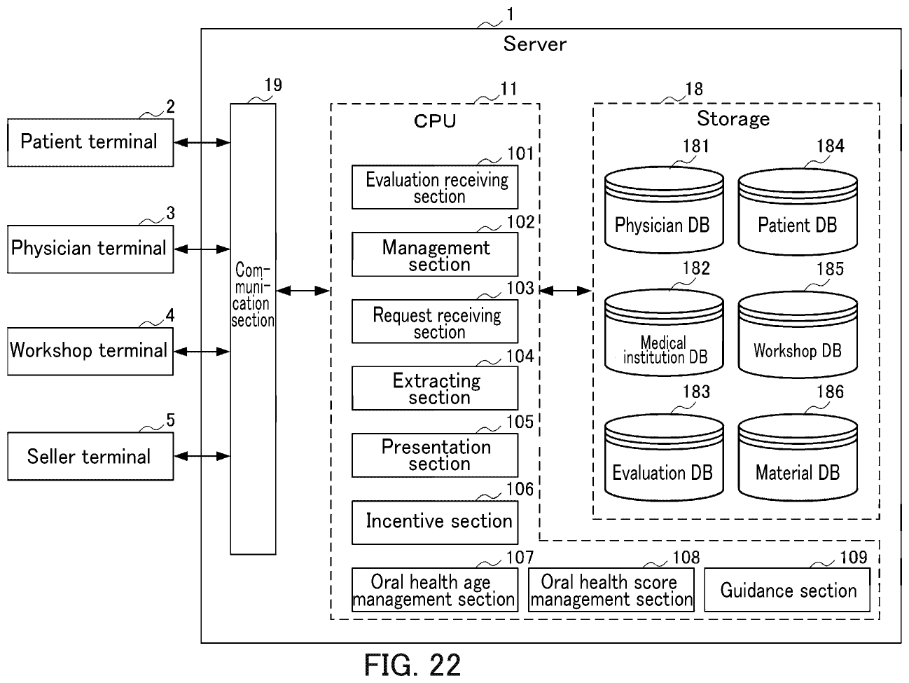

FIG. 22 is a functional block diagram illustrating an example of a functional configuration for implementing guidance support processing within the functional configuration of a server in an information processing system according to the second embodiment.

Note that the configuration of the information processing system including the server and the hardware configuration of the server according to the second embodiment of the information processing device of the present invention are respectively the same as those in FIGS. 12 and 13 according to the first embodiment, and description thereof is therefore omitted.

“Guidance support processing” refers to processing that supports the guidance of a patient U in receiving an examination at the medical facility H (dental clinic).

As illustrated in FIG. 22, when the guidance support processing is executed in the CPU 11 (FIG. 13) of the server 1, the CPU 11 may function as an oral health age management section 107, an oral health score management section 108, and a guidance section 109 in addition to the functional configuration in FIG. 14.

Assuming that the physician D has examined the teeth of the patient U, the oral health age management section 107 manages the dental health of the patient U according to the oral health age of the patient U indicated by the physician D.

The oral health score management section 108 manages the dental health of the patient U according to the oral health score given to the patient U according to self-care practiced by the patient U.

The guidance section 109 performs control to send a notification or email to the patient U as guidance for the patient U to receive examination from the physician D according to the oral health score given to the patient U.

The guidance section 109 also presents information indicating the content of risk (e.g., possibility of rheumatism) that the patient U will be exposed to if the oral health age of the patient U exceeds a certain level in the guidance provided to the patient U.

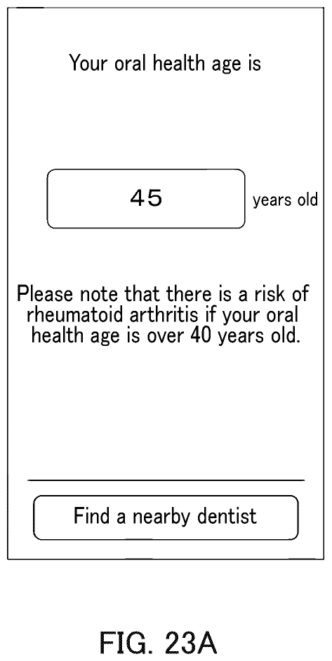

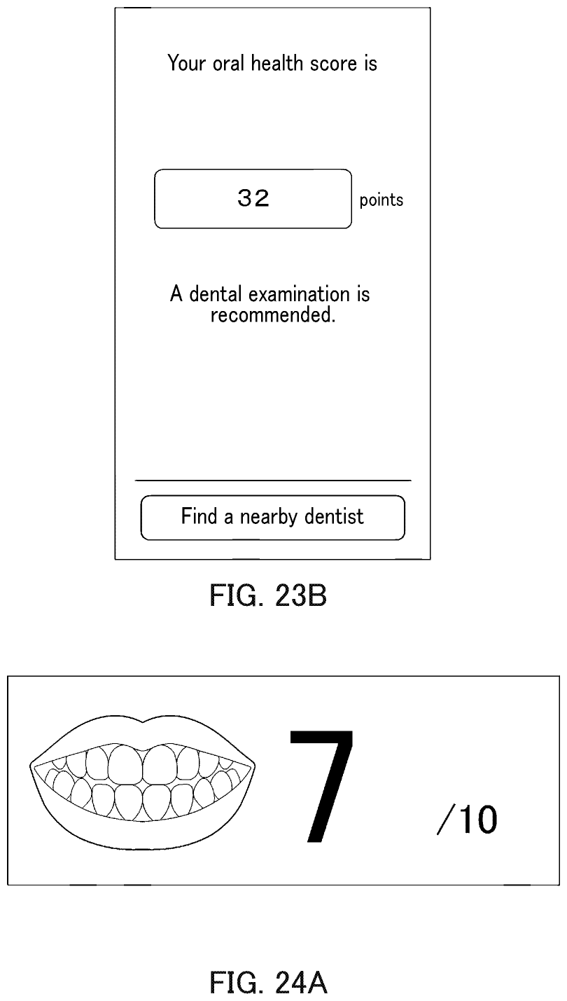

FIGS. 23A and 23B are diagrams illustrating respective examples of an oral health age inquiry screen and an oral health score inquiry screen displayed by a patient terminal as a specific example of the service in FIG. 21.

For a specific example, as illustrated in FIG. 23A, “Your oral health age is 45 years old” is displayed by the patient terminal 2 when the patient U operates the patient terminal 2 to try to confirm the oral health age of the patient U. In addition, a message such as “Please note that there is a risk of rheumatoid arthritis if your oral health age is over 40 years old” is added as content of the risk that the patient U may be exposed to according to the displayed health age. Through the above, the patient U is predicted to think about seeking an examination by the physician D. As such, as illustrated in FIG. 23A, the patient terminal 2 displays a button labeled “Find a nearby dentist” along with the oral health age and a predetermined message. When the patient U presses the button, the screen in FIG. 8B described above is displayed. That is, the operation of pressing the button labeled “Find a nearby dentist” provided in the screen illustrated in FIG. 23A is the same as the operation of pressing the button labeled “Find a nearby dentist” provided in the display region F2 in FIG. 8A described above.

For another example, as illustrated in FIG. 23B, “Your oral health score is 32” is displayed by the patient terminal 2 when the patient U operates the patient terminal 2 to try to confirm the oral health score of the patient U. In addition, a message such as “A dental examination is recommended.” is added as guidance to the patient U according to the displayed health score. Through the above, the patient U is predicted to think about receiving a dental examination. As such, as illustrated in FIG. 23B, the patient terminal 2 displays a button labeled “Find a nearby dentist” in the same manner as in FIG. 23A along with the oral health age and the message to guide the patient U. When the patient U presses the button, the screen in FIG. 8B described above is displayed. That is, the operation of pressing the button labeled “Find a nearby dentist” provided in the screen illustrated in FIG. 23B is the same as the operation of pressing the button labeled “Find a nearby dentist” provided in the display region F2 in FIG. 8A described above.

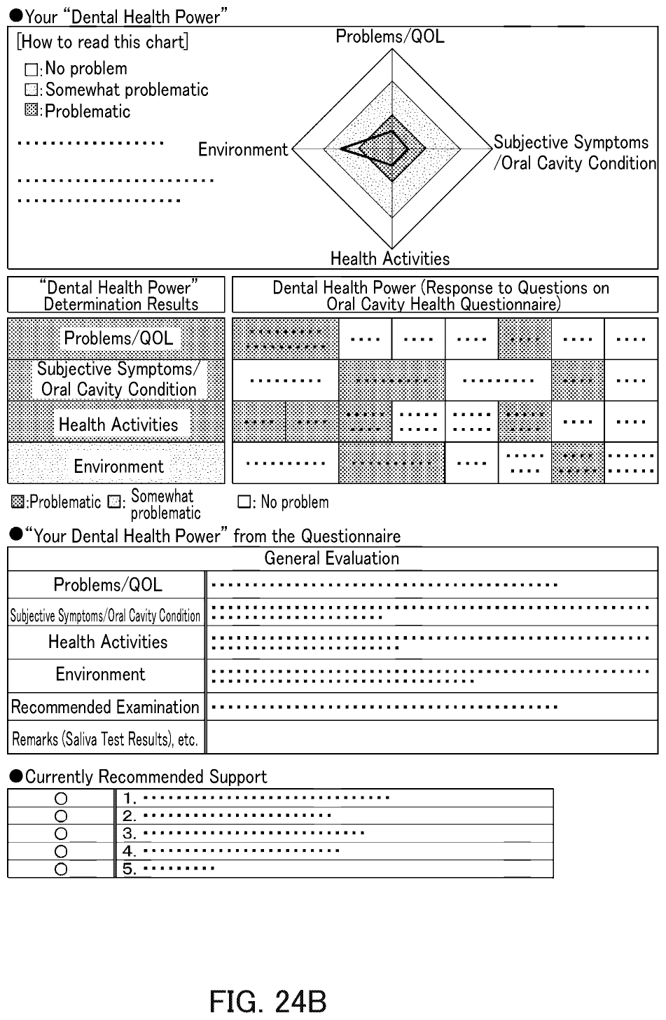

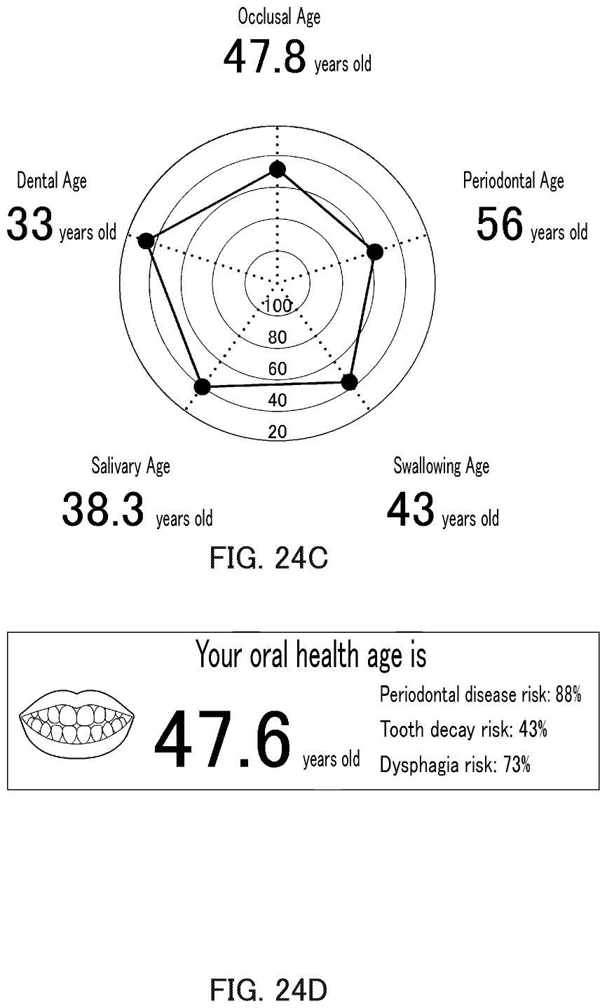

Next, a specific example of determination of “oral health age” by a physician D is described with reference to FIGS. 24A to 24C.

The present service implements an oral health (health) age measurement application (referred to in the following as an “age measurement app”) with the object of (1) providing health care related to eating and swallowing functions, (2) promoting visits by users of the patient app, (3) guiding young people, and (4) increasing the rate of regular examinations at a dental clinic.

Through the age measurement app, a simple diagnostic function can grant “awareness” of the importance of oral cavity function and oral cavity environment. A patient can also be guided to receive an oral cavity checkup by a physician D or a dental hygienist, which cannot be performed by self-checkup alone. The motivation of the patient U can also be expected to be increased or maintained.

The flow of the “oral health age diagnosis” by the age measurement app is as follows.

First, the patient U answers a questionnaire (STEP 1). Next, the patient U undergoes a determination of oral cavity function, oral cavity environment, and lifestyle (STEP 2). Through the above, simple visualizations of the determination results are provided. For example, FIG. 24A illustrates that a simple determination result by the physician D is 7 out of 10 points. Note that the “simple determination result” referred to here is not expressed in terms of age (oral health age), but rather as a score display. Through the above, it can be clarified that the patient U needs to receive a more detailed medical interview, testing, results, and treatment in order to calculate an oral health age instead of a score display. As a result, the patient U can be guided to an affiliated clinic.

Next, the patient U is guided to a dental clinic for diagnosis and regular examination (STEP 3). That is, opportunities are created for the patient U to have a stronger interest in oral cavity function and oral cavity environment regardless of the score in STEP 2. In other words, guidance to an affiliated dental clinic is implemented in order to evaluate the score of each item in more detail and calculate an “oral health age”. The service described above can also be used to lead or guide to the clinic.

Next, examination, diagnosis, and treatment are performed at a dental clinic (STEP 4). The content of the examination by a physician D is a score evaluation of “dental age”, “occlusal age”, “periodontal age”, “swallowing age”, and “salivary age” based on (1) the number of teeth, (2) tooth decay, (3) periodontal disease (periodontal pocket check), (4) saliva testing (measurement of volume and composition), (5) eating and swallowing function, (6) x-rays, and the answers to the questionnaire. Through the above, more detailed results of dental examination can be created. The results of the dental examination can also be visualized through radar charts or the like. For example, FIG. 24C illustrates a specific example of score evaluations for “dental age”, “occlusal age”, “periodontal age”, “swallowing age”, and “salivary age”.

Furthermore, FIG. 24B illustrates items such as a radar chart consisting of “Problems/Quality of Life (QOL),” “Environment,” “Health Activities,” and “Subjective Symptoms/Oral Cavity Condition”, and determination results of “dental health power” as “Your ‘Dental Health Power’”. Furthermore, a general evaluation is shown as “‘Your Dental Health Power’ from the Questionnaire”. A plurality of support measures is also illustrated as “Currently Recommended Support”.

The risk of periodontal disease, tooth decay, dysphagia, and the like can also be calculated.

FIG. 24D illustrates an example of calculation of oral health age, periodontal disease risk, tooth decay risk, and dysphagia risk. Through the above, the patient U is guided to a physician D at an affiliated clinic when more specialized treatment is necessary. When the oral health age related to the eating and swallowing function is found to differ from the actual age by a certain value or higher, it can also be linked to the functions of an “Oral Health Care Related to Eating and Swallowing Function” application.

Thereafter, continuous follow-up and treatment by a physician D or a dental hygienist are performed. By using it for general treatment and preventative dentistry (regular examination and recall), it is valuable as a tool linked to increasing the examination rate and recall rate.

Embodiments of the present invention have been described so far, but the present invention is not limited to the above embodiments. Variations, improvements, and the like within the scope in which the object of the present invention can be achieved are included in the present invention.

For example, in the embodiments described above, the physician D is a dentist, but this is only an example. The physician D need only be a physician in a position to provide some kind of medical care to a patient U. That is, a physician in the present invention is not limited to a dentist but is a concept that includes any physician including a surgeon, an internist, an ophthalmologist, and a dermatologist.

For another example, the “request” of the patient U in the embodiments described above is merely an example, and any item that the patient U may desire from the physician D or the medical institution can be a “request”.

For another example, the items to be entered in the check box input field of the evaluation content illustrated in FIG. 10(A) include not only negative evaluation content such as “I felt that the treatment was painful or inadequate”, “The explanation was insufficient”, “The customer service attitude was not good”, “I had to wait longer than necessary”, and “They did not listen to me”. For example, positive evaluation content such as “The treatment was not painful and I felt it was good”, “I was satisfied with the explanation”, “The customer service attitude was good”, “There was almost no waiting time”, and “They listened to me well” may be displayed.

For another example, in the embodiments described above, the subject of the usage suspension measures taken by the service provider G is a patient U or a physician D. However, a workshop or a material seller subject to evaluation by the physician D can also be included as a subject of the usage suspension measures.

For another example, in the embodiments described above, users of the present services include a patient U, a physician D, a workshop, and a material seller, but are not limited to these. For example, a nurse, a dental hygienist, a dental assistant, and any other person engaged in a medical institution can be a user of the present services. For a specific example, a nurse or a dental hygienist can receive provision of the services for physicians described above.

In other words, the physician D in the embodiments described above is only an example, and the present services can also target occupations other than physicians.

For a specific example, the present services can target hairdressers, craftsmen, and any other type of person who works in an organization and uses their individual skills.

For another example, although the embodiments described above assume that the patient U finds a desired physician D, the present services also allow the physician D to find a desired patient U using the physician app. The present services also allow the physician D to find a medical care location along with the desired patient U.

In addition, in the embodiments described above, points are given to the evaluation of the physician D or the like by the user U. However, the object of the points may be a response to a questionnaire by the user U. Note that the response to such a questionnaire can also be grasped as a type of “evaluation”. That is, the information processing system to which the present invention is applied can give points to any evaluation given by the user U.

For another example, in the embodiments described above, the physician D indicates an “oral health age” at each examination, but this is not a limitation. The timing at which the “oral health age” is indicated is not particularly limited. For example, after the “oral health age” is indicated in the first examination, the “oral health age” may be indicated at the time of the last examination after treatment has been completed through multiple examinations.

Furthermore, the system configuration illustrated in FIG. 6 and the hardware configuration of the server 1 illustrated in FIG. 7 are merely examples for achieving the object of the present invention and are not particular limitations.

The functional block diagrams illustrated in FIGS. 8 and 22 are merely examples and not particular limitations. That is, it is sufficient for the information processing system to include a function capable of executing the series of processing described above as a whole, and the sort of functional blocks used to implement this function is not particularly limited to FIGS. 8 and 22.

Also, the locations of the functional blocks are not limited to FIGS. 8 and 22 but may be any locations. For example, at least some of the functions on the server 1 side may be provided on the patient terminal 2 side or the physician terminal 3 side, or at least some of the functions of the patient terminal 2 side or the physician terminal 3 side may be provided on the server 1 side.

Furthermore, one functional block may be configured as a hardware unit, a software unit, or a combination of the two.

For example, in the embodiments described above, processing (a function) executed by the patient terminal 2 or the physician terminal 3 using application software may be processing (a function) executed by the server 1. In this case, the patient terminal 2 or the physician terminal 3 can obtain the result of the processing by accessing the server 1 using a browser or the like.

When the processing of each functional block is executed by software, the program that constitutes the software is installed to a computer or the like from a network or a recording medium.

The computer may be a computer that is embedded in dedicated hardware. The computer may also be a computer capable of executing various functions by installing various programs, such as a server as well as a generic smartphone or personal computer.

A recording medium containing such programs is not only composed of removable media distributed separately from a device main body to provide the programs to each user, but is also composed of a recording medium or the like that is provided to each user with the programs pre-embedded in the device main body.

Note that in the present specification, the steps describing a program recorded on a recording medium include not only processing performed sequentially in chronological order, but also processing performed in parallel or individually and not necessarily being processed in chronological order.

Furthermore, in the present specification, the term “system” means an overall device composed of a plurality of devices, a plurality of steps, or the like.

In summary, it is sufficient for the information processing device to which the present invention is applied to have the following configuration, which may be implemented in various embodiments.

That is, the information processing device to which the present invention is applied includes:

a first management means (e.g., the oral health age management section 107 in FIG. 22) which manages dental health of a patient according to a first evaluation result (e.g., the oral health age of a patient U as indicated by a physician D in FIG. 21) based on a first index assuming predetermined treatment (e.g., dental examination of the patient U by the physician D in FIG. 21) to improve the dental health of the patient (e.g., the patient U in FIG. 21);

a second management means (e.g., the oral health age management section 108 in FIG. 22) which manages the dental health of the patient according to a second evaluation result (e.g., an oral health score given to the patient U according to the self-care practiced by the patient U in FIG. 21) based on a second index;

and a guidance means (e.g., the guidance section 109 in FIG. 20) which performs predetermined guidance (e.g., the notification or email described above) for the patient to perform the predetermined treatment (e.g., dental examination of the patient U by the physician D in FIG. 21) to improve the dental health of the patient according to the second evaluation result.

Through the above, the dental health of the patient is managed according to each of the first evaluation result based on the first index assuming the predetermined treatment to improve the dental health of the patient and the second evaluation result based on the second index. According to the second evaluation result, predetermined guidance is performed for the patient so that the predetermined treatment is performed to improve the dental health of the patient.

As a result, for example, a dentist can guide the patient to an examination and the patient who has received examination by the dentist can efficiently perform self-care.

The first index can also be an index of the level of dental health of the patient expressed in terms of age.

Through the above, the patient can compare the age of the patient to the dental age of the patient (e.g., the oral health age described above). Thus, the patient can easily imagine the level of dental health of the patient.

Furthermore, the guidance means

can present, in the guidance performed for the patient, information indicating the content of risk (e.g., possibility of rheumatism) that the patient will be exposed to when the age of the patient is a certain level or higher.

Through the above, the patient can easily imagine what the current dental health risks are for the patient.

### REFERENCE SIGNS LIST

1 Server
2, 2-1 to 2-n Patient terminal
3, 3-1 to 3-m Physician terminal
4 Workshop terminal
5 Material seller terminal
11 CPU
12 ROM
13 RAM
14 Bus
15 Input/output interface
16 Input section
17 Output section
18 Storage
19 Communication section
20 Drive
30 Removable media
101 Evaluation receiving section
102 Management section
103 Request receiving section
104 Extracting section
105 Presentation section
106 Incentive section
107 Oral health age management section
108 Oral health score management section
109 Guidance section
181 Physician DB
182 Medical institution DB
183 Evaluation DB
184 Patient DB
185 Workshop DB
186 Material DB
U, U1 to Un Patient
D, D1 to Dm Physician
F Display region
N Network

1. An information processing device comprising:
a first management means configured to manage dental health of a patient according to a first evaluation result based on a first index assuming predetermined treatment to improve the dental health of the patient;
a second management means configured to manage the dental health of the user according to a second evaluation result based on a second index; and
a guidance means configured to perform predetermined guidance for the patient to perform the predetermined treatment to improve the dental health of the patient according to the second evaluation result.

2. The information processing device according to claim 1, wherein
the first index is an index of a level of the dental health of the patient expressed in terms of age.

3. The information processing device according to claim 2, wherein
the guidance means presents, in the guidance performed for the patient, information indicating content of risk that the patient will be exposed to when the age is a certain level or higher.
